(19) United States
(12) Patent Application Publication
(10) Pub. No.: US 2006/0286064 A1
(43) Pub. Date: Dec. 21, 2006

(54) Therapeutic polymers and methods

(75) Inventors: William G. Turnell, San Diego, CA (US); Zaza D. Gomurashvili, La Jolla, CA (US); Ramaz Katsarava, Tbilisi, GA (US)

Correspondence Address: DLA PIPER US LLP, 4365 EXECUTIVE DRIVE, SUITE 1100, SAN DIEGO, CA, 92121-2133, US

(73) Assignee: MediVas, LLC, San Diego, CA (US)

(21) Appl. No.: 11/446,405

(22) Filed: Jun. 2, 2006

Related U.S. Application Data
(63) Continuation-in-part of application No. 10/362,848, filed on Oct. 14, 2003.
Application No. 10/362,848 is a continuation of application No. 09/651,338, filed on Aug. 30, 2000, now Pat. No. 6,503,538.
(60) Provisional application No. 60/687,570, filed on Jun. 3, 2005.
(60) Provisional application No. 60/759,179, filed on Jan. 13, 2006.

(51) Int. Cl. A61K 31/785; C08F 20/00

(52) U.S. Cl. 424/78.27; 525/437; 525/440

(57) ABSTRACT

The present invention provides biodegradable therapeutic polymer compositions based on poly(ester amide) (PEA), poly(ester urethane) (PEUR), and poly(ester urea) (PEU) polymers useful for in vivo delivery of at least one therapeutic diol or di-acid incorporated into the backbone of the biodegradable polymer. The therapeutic polymer compositions biodegrade in vivo by enzymatic action to release therapeutic diols or di-acids from the polymer backbone in a controlled manner over time. The invention compositions are stable, can be lyophilized for transportation and storage, and can be redispersed for administration. Due to structural properties of the PEA and PEUR polymers used, the invention therapeutic polymer compositions provide for high loading of the therapeutic diol or di-acid, as well as optional bioactive agents.

### RELATED APPLICATIONS

This application relies for priority under 35 U.S.C. §119(e) on U.S. Ser. Nos. 60/687,570, filed Jun. 3, 2005 and 60/759,179, filed Jan. 13, 2006 and this application is a continuation in part under 35 U.S.C. §120 of U.S. Ser. No. 10/362,848, filed Oct. 14, 2003, which is a continuation of U.S. Pat. No. 6,503,538 B1, each of which is incorporated herein by reference.

### FIELD OF THE INVENTION

The invention relates, in general, to drug delivery systems and, in particular, to polymer delivery compositions that incorporate a therapeutic agent into a biodegradable polymer backbone.

### BACKGROUND INFORMATION

The earliest drug delivery systems, first introduced in the 1970s, were based on polymers formed from lactic and glycolic acids. Today, polymeric materials still provide the most important avenues for research, primarily because of their ease of processing and the ability of researchers to readily control their chemical and physical properties via molecular synthesis. Basically, two broad categories of polymer systems, both known as “microspheres” because of their size and shape, have been studied: reservoir devices and matrix devices. The former involves the encapsulation of a pharmaceutical product within a polymer shell, whereas the latter describes a system in which a drug is physically entrapped within a polymer network.

The release of medications from either category of polymer device traditionally has been diffusion-controlled. Currently, however, modern research is aimed at investigating biodegradable polymer systems. These drug deliverers degrade into biologically acceptable compounds, often through the process of hydrolysis, and leave their incorporated medications behind. This erosion process occurs either in bulk (wherein the matrix degrades uniformly) or at the polymer's surface (whereby release rates are related to the polymer's surface area). The degradation process itself involves the breakdown of these polymers into lactic and glycolic acids. These acids are eventually reduced by the Kreb's cycle to carbon dioxide and water, which the body can easily expel.

Regular AA-BB type amino acid based bio-analogous poly(ester amides) (PEAs) and poly(ester urethanes) (PEURs) consisting of nontoxic building blocks, such as hydrophobic α-amino acids, α,ω-diols, and aliphatic dicarboxylic acids have been investigated as biomaterials for drug release and tissue engineering applications (G. Tsitlanadze et al. J. Biomater. Sci. Polymer Edn, (2004) 15: 1-24). The combination of controlled enzymatic degradation and low nonspecific hydrolysis rates of PEAs and PEURs make them attractive for drug delivery applications. In particular, these polymers appear to be blood and tissue compatible with advantageous properties for cardiovascular applications (K. DeFife et al. Transcatheter Cardiovascular Therapeutics—TCT 2004 Conference. Poster presentation. Washington D.C. (2004)).

In most drug-eluting applications, the drug is physically matrixed by dissolving or melting with a polymer. Another approach has also been reported in which a drug is chemically attached as a side group to a polymer.

If a drug or other therapeutic agent is covalently incorporated into a biodegradable polymer, a therapeutic polymer is formed. Such compositions represent synthetic polymers that combine therapeutic or palliative bioactivity with desirable mechanical and physical properties, and degrade into useful therapeutic active compounds. In other words, the compositions have the activity of a drug, but have the physical properties of a material. Recently, new therapeutic polyesters, polyamides, and poly(ester anhydrides) were reported, wherein non-steroidal anti-inflammatory drugs (NSAIDs) were incorporated into a polymer backbone (R. C. Schmeltzer et al. Biomacromolecules. (2005) 6(1):359-367). In such compositions, drug release is directly dependent on the hydrolytic or enzymatic cleavage of polymer-drug binding groups. One of the advantages of a “backbone as a drug” polymer is that a high amount of drug or therapeutic compound can be incorporated into the structure.

Thus, there is a need in the art for more and better compositions and methods for incorporating therapeutic molecules, such as drugs and other bioactive agents, into the backbones of polymers for use in polymer delivery systems in which controlled rate of therapeutic release is combined with desirable mechanical and physical properties.

Finally, recent research has shown that hydrogel-type materials can be used to shepherd various medications through the stomach and into the more alkaline intestine. Hydrogels are cross-linked, hydrophilic, three-dimensional polymer networks that are highly permeable to entrap molecules, which can be released in vivo through their weblike surfaces. Depending on the chemical composition of the gel, different internal and external stimuli (e.g., changes in pH, application of a magnetic or electric field, variations in temperature, and ultrasound irradiation) may be used to trigger the swelling effect. Once triggered, however, the rate of entrapped drug release is generally determined by the cross-linking level of the polymer network.

Thus, a need exists in the art for new and better compositions and methods of use for biodegradable polymer compositions for delivering therapeutic molecules, such as drugs and other bioactive agents. Particularly, the need exists for new and better delivery compositions that incorporate a therapeutic agent into the backbone of a polymer for time release of the therapeutic agent in a consistent and reliable manner.

### SUMMARY OF THE INVENTION

The present invention is based on the premise that poly(ester amide) (PEA), poly(ester urethane) (PEUR), and poly(ester urea) (PEU) polymers, can be formulated as polymer delivery compositions that incorporate a therapeutic diol or di-acid into the backbone of the polymer for time release of the therapeutic agent in a consistent and reliable manner by biodegradation of the polymer.

In one embodiment, the invention provides a biodegradable therapeutic polymer composition in which at least one therapeutic diol or di-acid is incorporated into the backbone of one or more biodegradable polymers. The biodegradable polymer of the composition contains or is a blend of at least one PEA having a structural formula described by structural formula (I), 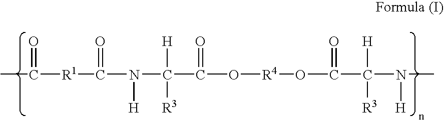
wherein n ranges from about 5 to about 150; R¹ is independently selected from residues of α,ω-bis(4-carboxyphenoxy)-(C₁-C₈) alkane, 3,3′-(alkanedioyldioxy)dicinnamic acid or 4,4′-(alkanedioyldioxy)dicinnamic acid, (C₂-C₂₀) alkylene, (C₂-C₂₀) alkenylene or saturated or unsaturated residues of therapeutic di-acids; the R³s in individual n monomers are independently selected from the group consisting of hydrogen, (C₁-C₆) alkyl, (C₂-C₆) alkenyl, (C₂-C₆) alkynyl, (C₆-C₁₀) aryl (C₁-C₆) alkyl, and —(CH₂)₂S(CH₃); and R⁴ is independently selected from the group consisting of (C₂-C₂₀) alkylene, (C₂-C₂₀) alkenylene, (C₂-C₈) alkyloxy (C₂-C₂₀) alkylene, bicyclic-fragments of 1,4:3,6-dianhydrohexitols of structural formula (II), saturated or unsaturated therapeutic diol residues, and combinations thereof; 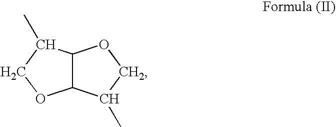
except that at least one of R¹ and R⁴ is a therapeutic amount of the residue of a therapeutic di-acid and diol, respectively,

or at least one PEA polymer having a chemical formula described by structural formula III: 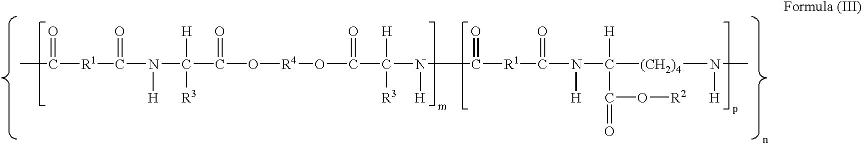
wherein n ranges from about 5 to about 150, m ranges about 0.1 to 0.9: p ranges from about 0.9 to 0. 1; wherein R¹ is independently selected from residues of a,(o-bis(4-carboxyphenoxy)-(C₁-C₈) alkane, 3,3′-(alkanedioyldioxy)dicinnamic acid or 4,4′-(alkanedioyldioxy)dicinnamic acid, (C₂-C₂₀) alkylene, (C₂-C₂₀) alkenylene or a saturated or unsaturated residues of therapeutic di-acids; each R² is independently hydrogen, (C₁-C₁₂) alkyl or (C₆-C₁₀) aryl or a protecting group; the R³s in individual m monomers are independently selected from the group consisting of hydrogen, (C₁-C₆) alkyl, (C₂-C₆) alkenyl, (C₂-C₆) alkynyl, (C₆-C₁₀) aryl (C₁-C₆) alkyl, —(CH₂)₂S(CH₃); and R⁴ is independently selected from the group consisting of (C₂-C₂₀) alkylene, (C₂-C₂₀) alkenylene, (C₂-C₈) alkyloxy (C₂-C₂₀) alkylene, bicyclic-fragments of 1,4:3,6-dianhydrohexitols of structural formula (II), residues of saturated or unsaturated therapeutic diols and combinations thereof, except that at least one of R¹ and R⁴ in at least one of the m monomers is the residue of a therapeutic di-acid or diol, respectively,

or at least one PEUR having a chemical formula described by general structural formula (IV), 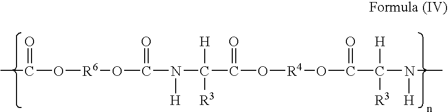
wherein n ranges from about 5 to about 150; wherein R³s in independently selected from the group consisting of hydrogen, (C₁-C₆) alkyl, (C₂-C₆) alkenyl, (C₂-C₆) alkynyl, (C₆-C₁₀) aryl (C₁-C₆) alkyl, and —CH₂)₂S(CH₃) and; R⁴ is selected from the group consisting of (C₂-C₂₀) alkylene, (C₂-C₂₀) alkenylene or alkyloxy, and bicyclic-fragments of 1,4:3,6-dianhydrohexitols of structural formula (II), or fragments of saturated or unsaturated therapeutic diols; and R⁶ is independently selected from (C₂-C₂₀) alkylene, (C₂-C₂₀) alkenylene or alkyloxy, bicyclic-fragments of 1,4:3,6-dianhydrohexitols of general formula (II), residues of saturated or unsaturated therapeutic diols, and combinations thereof, except that the R⁴ and R⁶ within at least one of the n unit is the residue of the therapeutic diol;

or at least one PEUR polymer having a chemical structure described by general structural formula (V) 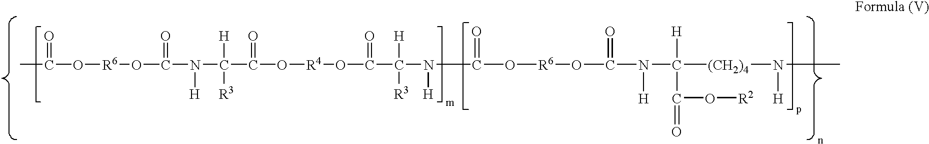
wherein n ranges from about 5 to about 150, m ranges about 0.1 to about 0.9: p ranges from about 0.9 to about 0.1; R² is independently selected from hydrogen, (C₆-C₁₀) aryl (C₁-C₆) alkyl, or a protecting group; the R³s in an individual m monomer are independently selected from the group consisting of hydrogen, (C₁-C₆) alkyl, (C₂-C₆) alkenyl, (C₂-C₆) alkynyl, (C₆-C₁₀) aryl(C₁-C₆) alkyl, and —CH₂)₂S(CH₃); R⁴ is selected from the group consisting of (C₂-C₂₀) alkylene, (C₂-C₂₀) alkenylene or alkyloxy, bicyclic-fragments of 1,4:3,6-dianhydrohexitols of structural formula (II) or fragments of saturated or unsaturated therapeutic diols; and R⁶ is independently selected from (C₂-C₂₀) alkylene, (C₂-C₂₀) alkenylene or alkyloxy, bicyclic-fragments of 1,4:3,6-dianhydrohexitols of general formula (II), a residue of a saturated or unsaturated therapeutic diols, and combinations thereof, except that the R⁴ or R⁶ within at least one of the m unit is the residue of a therapeutic diol,

or at least one PEU polymer having a chemical formula described by general structural formula (VI): 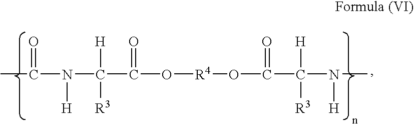
wherein n is about 10 to about 150; each R³s within an individual n unit are independently selected from hydrogen, (C₁-C₆) alkyl, (C₂-C₆) alkenyl, (C₂-C₆) alkynyl, (C₆-C₁₀) aryl (C₁-C₆)alkyl, and —CH₂)₂S(CH₃); R⁴ is independently selected from (C₂-C₂₀) alkylene, (C₂-C₂₀) alkenylene, (C₂-C₈) alkyloxy (C₂-C₂₀) alkylene, a residue of a saturated or unsaturated therapeutic diol; or a bicyclic-fragment of a 1,4:3,6-dianhydrohexitol of structural formula (II), and combinations thereof, except that the R⁴ within at least one of the n units is the residue of a therapeutic diol;

or at least one PEU having a chemical formula described by structural formula (VII) 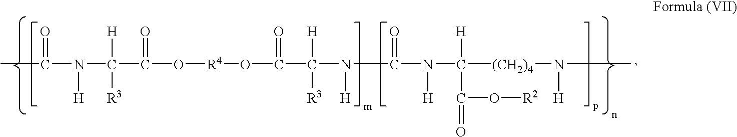
wherein m is about 0.1 to about 1.0; p is about 0.9 to about 0. 1; n is about 10 to about 150; each R2 is independently hydrogen, (C₁-C₁₂) alkyl or (C₆-C₁₀) aryl; the R³s within an individual m monomer are independently selected from hydrogen, (C₁-C₆) alkyl, (C₂-C₆) alkenyl, (C₂-C₆) alkynyl, (C₆-C₁₀) aryl (C₁-C₆) alkyl, and —CH₂)₂S(CH₃); each R⁴ is independently selected from (C₂-C₂₀) alkylene, (C₂-C₂₀) alkenylene, (C₂-C₈) alkyloxy (C₂-C₂₀) alkylene, a residue of a saturated or unsaturated therapeutic diol; or a bicyclic-fragment of a 1,4:3,6-dianhydrohexitol of structural formula (II), and combinations thereof, except that the R⁴ in at least one of the m units is the residue of a therapeutic diol.

In another embodiment, the invention provides methods for administering a therapeutic diol or di-acid to a subject by administering to the subject an invention therapeutic polymer composition containing one or more polymers of formula(s) (I) or (III)-(VII) in the form of a liquid dispersion, which composition biodegrades by enzymatic action to release the therapeutic diol or di-acid over time.

In yet another embodiment, the invention provides a bis-nucleophilic compound wherein the compound is a di(amino acid)-estradiol-3,17-β-diester, or salt thereof.

### A BRIEF DESCRIPTION OF THE FIGURES

FIG. 1 is showing a ¹H NMR (500 MHz, DMSO-d₆) spectrum of 17β-estradiol based monomer (compound 5 of Example 1).

FIG. 2 is a trace of differential scanning calorimetry (DSC) of the therapeutic PEA polymer formed in Example 1, showing a first heating curve, with sharp melting endotherm.

FIG. 3 is showing a ¹H NMR (500 MHz, DMSO-d₆) spectrum of an invention 17β-estradiol-based PEA copolymer (scheme 5) formed in Example 1.

### DETAILED DESCRIPTION OF THE INVENTION

The invention is based on the discovery that biodegradable poly(ester amide) (PEA), and poly(ester urethane) (PEUR) polymers can be used to create a therapeutic polymer composition for in vivo delivery of at least one therapeutic diol or di-acid contained within a biodegradable polymer backbone. The therapeutic PEA, PEUR and PEU polymer compositions biodegrade in vivo by enzymatic action at the surface so as to release therapeutic diols or di-acids from the polymer backbone in a controlled manner over time. The invention compositions are stable, and can be lyophilized for transportation and storage and can be redispersed for administration. Due to structural properties of the PEA and PEUR polymers used, the invention therapeutic polymer compositions provide for high loading of the therapeutic diol or di-acid, as well as optional bioactive agents.

As used herein, a “therapeutic diol or di-acid” means any diol or di-acid molecule, whether synthetically produced, or naturally occurring (e.g., endogenously) that affects a biological process in a mammalian individual, such as a human, in a therapeutic or palliative manner when administered to the mammal.

As used herein, the term “residue of a therapeutic di-acid” means a portion of such a therapeutic di-acid, as described herein, that excludes the two carboxyl groups of the di-acid. As used herein, the term “residue of a therapeutic diol” means a portion of a therapeutic diol, as described herein, that excludes the two hydroxyl groups of the diol. The corresponding therapeutic di-acid or diol containing the “residue” thereof is used in synthesis of the polymer compositions. The residue of the therapeutic di-acid or diol is reconstituted in vivo (or under similar conditions of pH, aqueous media, and the like) to the corresponding diol or di-acid upon release from the backbone of the polymer by biodegradation in a controlled manner that depends upon the properties of the particular PEA, PEUR or PEU polymer selected to fabricate the composition, which properties are well known in the art and as described herein, for example in the Examples.

As used herein the term “bioactive agent” means a bioactive agent as disclosed herein that is not incorporated into the polymer backbone. One or more such bioactive agents may optionally be dispersed in the invention therapeutic polymer compositions. As used herein, the term “dispersed” is used to refer to bioactive agents (not incorporated into the polymer backbone) and means that the bioactive agent is dispersed, mixed, dissolved, homogenized, and/or covalently bound (“dispersed”) in a polymer, for example attached to a functional group in the polymer of the composition or to the surface of a polymer particle, but not incorporated into the backbone of a PEA or PEUR polymer. To distinguish backbone-incorporated therapeutic diols and di-acids from those that are not incorporated into the polymer backbone, (as a residue thereof), such dispersed therapeutic diols and di-acids are referred to herein as “bioactive agent(s)” and may be contained within polymer conjugates or otherwise dispersed in the polymer composition in the same manner as other bioactive agents, as described below.

The term, “biodegradable” as used herein to describe the polymers used in the invention therapeutic polymer compositions means the polymer is capable of being broken down into innocuous and therapeutic products in the normal functioning of the body. The polymers in the invention therapeutic polymer compositions include hydrolyzable ester and enzymatically cleavable amide linkages that provide biodegradability, and are typically chain terminated predominantly with amino groups. Thus, in the case of a naturally occurring therapeutic diol or di-acid, the breakdown product delivered is the naturally occurring molecule. Optionally, these amino termini can be acetylated or otherwise capped by conjugation to any other acid-containing, biocompatible molecule, to include without restriction organic acids, bioinactive biologics, and bioactive agents as described herein. In one embodiment, the entire therapeutic polymer composition is biodegradable.

More particularly, the invention therapeutic polymer composition comprises a biodegradable, biocompatible polymer with a residue of at least one therapeutic diol or di-acid incorporated into the backbone of the polymer. In one embodiment, the invention therapeutic polymer composition comprises at least one PEA having a chemical formula described by structural formula (I), 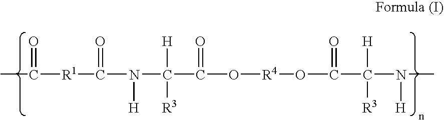
wherein n ranges from about 5 to about 150; R¹ is independently selected from residues of 3,3′-(alkanedioyldioxy)dicinnamic acid or 4,4′-(alkanedioyldioxy)dicinnamic acid or of α,ω-bis(4-carboxyphenoxy)-(C₁-C₈) alkane, (C₂- C₂₀) alkylene, (C₂-C₂₀) alkenylene or residues of saturated or unsaturated therapeutic di-acids; the R³s in individual n units are independently selected from the group consisting of hydrogen, (C₁-C₆) alkyl, (C₂-C₆) alkenyl, (C₂-C₆) alkynyl, (C₆-C₁₀) aryl (C₁-C₆) alkyl, and —CH₂)₂S(CH₃); and R⁴ is independently selected from the group consisting of (C₂-C₂₀) alkylene, (C₂-C₂₀) alkenylene, (C₂-C₈) alkyloxy (C₂-C₂₀) alkylene, bicyclic-fragments of 1,4:3,6-dianhydrohexitols of structural formula (II), saturated or unsaturated therapeutic diol residues, and combinations thereof; 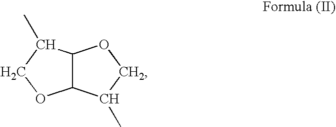
except that at least one of R¹ and R⁴ is a therapeutic amount of the residue of a therapeutic di-acid or diol, respectively;

or at least one PEA polymer having a chemical formula described by structural formula (III): 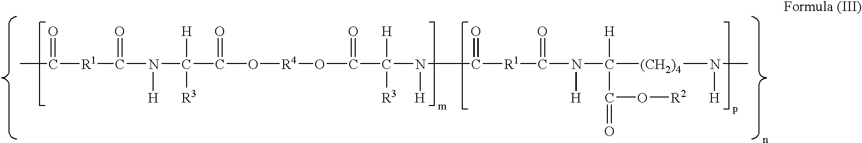
wherein n ranges from about 5 to about 150, m ranges about 0.1 to 0.9; p ranges from about 0.9 to 0.1; wherein R¹ is independently selected from residues of x,(o-bis(4-carboxyphenoxy)-(C₁-C₈) alkane, 3,3′-(alkanedioyldioxy)dicinnamic acid or 4,4′-(alkanedioyldioxy)dicinnamic acid, (C₂-C₂₀) alkylene, (C₂-C₂₀) alkenylene or a saturated or unsaturated residues of therapeutic di-acids; each R² is independently hydrogen, (C₁-C₁₂) alkyl or (C₆-C₁₀) aryl or a protecting group; the R³s in individual m monomers are independently selected from the group consisting of hydrogen, (C₁-C₆) alkyl, (C₂-C₆) alkenyl, (C₂-C₆) alkynyl, (C₆-C₁₀) aryl (C₁-C₆) alkyl, and —(CH₂)₂S(CH₃); and R⁴ is independently selected from the group consisting of (C₂-C₂₀) alkylene, (C₂-C₂₀) alkenylene, (C₂-C₈) alkyloxy (C₂-C₂₀) alkylene, bicyclic-fragments of 1,4:3,6-dianhydrohexitols of structural formula (II), residues of saturated or unsaturated therapeutic diols and combinations thereof; except that at least one of R¹ and R⁴ in at least one of the m units is the residue of a therapeutic di-acid or diol, respectively;

or at least one PEUR having a chemical formula described by general structural formula (IV), 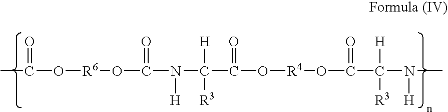
wherein n ranges from about 5 to about 150; wherein R³s in independently selected from the group consisting of hydrogen, (C₁-C₆) alkyl, (C₂-C₆) alkenyl, (C₂-C₆) alkynyl, (C₆-C₁₀) aryl(C₁-C₆) alkyl, and —(CH₂)₂S(CH₃) and; R⁴ is selected from the group consisting of (C₂-C₂₀) alkylene, (C₂-C₂₀) alkenylene or alkyloxy, and bicyclic-fragments of 1,4:3,6-dianhydrohexitols of structural formula (II), residues of saturated or unsaturated therapeutic diols and combinations thereof; and R⁶ is independently selected from (C₂-C₂₀) alkylene, (C₂-C₂₀) alkenylene or alkyloxy, bicyclic-fragments of 1,4:3,6-dianhydrohexitols of general formula (II), residues of saturated or unsaturated therapeutic diols, and combinations thereof, except that the R⁴ and/or R⁶ within at least one of the n units is the residue of the therapeutic diol;

or at least one PEUR polymer having a chemical structure described by general structural formula (V) 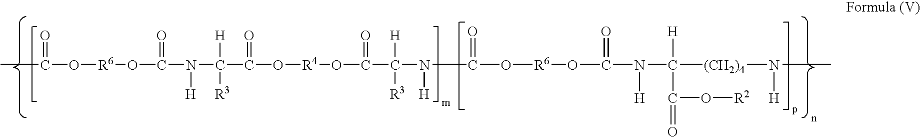
wherein n ranges from about 5 to about 150, m ranges about 0.1 to about 0.9: p ranges from about 0.9 to about 0.1; R²,is independently selected from hydrogen, (C₆-C₁₀) aryl (C₁-C₆) alkyl, or a protecting group; the R³s in an individual m units are independently selected from the group consisting of hydrogen, (C₁-C₆) alkyl, (C₂-C₆) alkenyl, (C₂-C₆) alkynyl, (C₆-C₁₀) aryl(C₁-C₆) alkyl, and —(CH₂)₂S(CH₃); R⁴ is selected from the group consisting of (C₂-C₂₀) alkylene, (C₂-C₂₀) alkenylene or alkyloxy, and bicyclic-fragments of 1,4:3,6-dianhydrohexitols of structural formula (II); and R⁶ is independently selected from (C₂-C₂₀) alkylene, (C₂-C₂₀) alkenylene or alkyloxy, bicyclic-fragments of 1,4:3,6-dianhydrohexitols of general formula (II), a residue of a saturated or unsaturated therapeutic diol, and combinations thereof, except that the R⁶ within at least one of the m units is the residue of a therapeutic diol;

or at least one PEU polymer having a chemical formula described by general structural formula (VI): 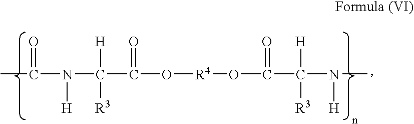
wherein n is about 10 to about 150; each R³s within an individual n unit are independently selected from hydrogen, (C₁-C₆) alkyl, (C₂-C₆) alkenyl, (C₂-C₆) alkynyl, (C₆-C₁₀) aryl (C₁-C₆)alkyl, and —(CH₂)₂S(CH₃); R⁴ is independently selected from (C₂-C₂₀) alkylene, (C₂-C₆) alkenylene, (C₂-C₈) alkyloxy (C₂-C₂₀) alkylene, a residue of a saturated or unsaturated therapeutic diol; or a bicyclic-fragment of a 1,4:3,6-dianhydrohexitol of structural formula (II), and combinations thereof, except that the R⁴ within at least one of the n units is the residue of a therapeutic diol;

or at least one PEU having a chemical formula described by structural formula (VII) 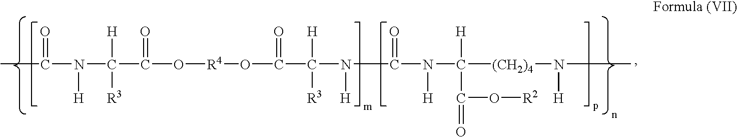
wherein m is about 0.1 to about 1.0; p is about 0.9 to about 0.1; n is about 10 to about 150; each R² is independently hydrogen, (C₁-C₁₂) alkyl or (C₆-C₁₀) aryl, or a protecting group; the R³s within an individual m unit are independently selected from hydrogen, (C₁-C₆) alkyl, (C₂-C₆) alkenyl, (C₂-C₆) alkynyl, (C₆-C₁₀) aryl (C₁-C₆)alkyl, and —(CH₂)₂S(CH₃); each R⁴ is independently selected from (C₂-C₂₀) alkylene, (C₂-C₂₀) alkenylene, (C₂-C₈) alkyloxy (C₂-C₂₀) alkylene, a residue of a saturated or unsaturated therapeutic diol; or a bicyclic-fragment of a 1,4:3,6-dianhydrohexitol of structural formula (II), and combinations thereof, except that the R⁴ in at least one of the m monomers is the residue of a therapeutic diol.

The bicyclic-fragments of such dianhydrohexitols can be derived from sugar alcohols, such as D-glucitol, D-mannitol and L-iditol. Dianhydrosorbitol is the presently preferred bicyclic fragment of a 1,4:3,6-dianhydrohexitol for use in the PEA, PEUR and PEU polymers used in fabrication of the invention therapeutic polymer compositions.

The protecting group can be t-butyl or any other protecting group known in the art.

In one embodiment, the residue of the therapeutic diol or di-acid incorporated into the polymer backbone of the invention therapeutic polymer composition of any one of Formulas (I) and (III)-(VII) is a therapeutic amount of the therapeutic diol or di-acid so that, upon administration, the composition biodegrades to release a therapeutic amount of the therapeutic diol or di-acid to the subject.

The invention therapeutic polymer compositions in which a therapeutic diol and/or di-acid is used in the place of a diol and/or di-acid otherwise useful in making PEA, PEUR, or PEU polymers as described herein, can be formulated into particles to provide a variety of properties. The particles can have a variety of sizes and structures suitable to meet differing therapeutic goals and routes of administration as described in full in co-pending U.S. provisional applications Nos. 60/654,715, filed Feb. 17, 2005, 60/684,670, filed May 25, 2005, and 60/737,401, filed Nov. 14, 2005.

As used herein, the terms “amino acid” and “α-amino acid” mean a chemical compound containing an amino group, a carboxyl group and a pendent R group, such as the R³ groups defined herein. As used herein, the term.“biological α-amino acid” means the amino acid(s) used in synthesis are selected from phenylalanine, leucine, glycine, alanine, valine, isoleucine, methionine, or a mixture thereof.

As used herein, a “therapeutic diol” or “therapeutic di-acid” means, respectively, any diol or di-acid molecule, whether synthetically produced, or naturally occurring (e.g., endogenously) that affects a biological process in a mammalian individual, such as a human, in a therapeutic or palliative manner when administered to the mammal.

As used herein, the term “residue of a therapeutic diol” means a portion of a therapeutic diol, as described herein, which portion excludes the two hydroxyl groups of the diol. As used herein, the term “residue of a therapeutic di-acid” means a portion of a therapeutic di-acid, as described herein, which portion excludes the two carboxyl groups of the di-acid. The corresponding therapeutic diol or di-acid containing the “residue” thereof is used in synthesis of the polymer compositions. The residue of the therapeutic di-acid or diol is reconstituted in vivo (or under similar conditions of pH, aqueous media, and the like) to the corresponding di-acid or diol upon release from the backbone of the polymer by biodegradation in a controlled manner that depends upon the properties of the PEA, PEUR or PEU polymer selected to fabricate the composition, which properties are as known in the art and as described herein.

As used herein the term “bioactive agent” means an active agent that affects a biological process in a mammalian individual, such as a human, in a therapeutic or palliative manner when administered to the mammal and that is not incorporated into the polymer backbone. Bioactive agents may include, without limitation, small molecule drugs, peptides, proteins, DNA, cDNA, RNA, sugars, lipids and whole cells. One or more such bioactive agents may be dispersed in the invention therapeutic polymer compositions.

As used herein, the term “dispersed” as used to refer to bioactive agents means that the bioactive agent is loaded into, mixed, dissolved, homogenized, and/or covalently bound (“dispersed”) in a polymer, for example, attached to a functional group in the therapeutic polymer of the composition or to the surface of a polymer particle, but not incorporated into the backbone of a PEA, PEUR, or PEU polymer. To distinguish dispersed therapeutic diols and di-acids from those that are incorporated into the polymer backbone, (as a residue thereof), such dispersed diols and di-acids are referred to herein as “bioactive agent(s)” and may be linked to the polymer, contained within polymer conjugates or otherwise dispersed in the invention therapeutic polymer composition the same as other bioactive agents disclosed herein.

The term, “biodegradable” as used herein to describe the invention therapeutic polymer compositions means the polymer used therein is capable of being broken down into innocuous products in the normal functioning of the body. This is particularly true when the amino acids used in fabrication of the therapeutic polymer compositions are biological L-α-amino acids. The polymers in the invention therapeutic polymer compositions include hydrolyzable ester and enzymatically cleavable amide linkages that provide biodegradability, and are typically chain terminated, predominantly with amino groups. Optionally, the amino termini of the polymers can be acetylated or otherwise capped by conjugation to any other acid-containing, biocompatible molecule, to include without restriction organic acids, bioinactive biologics, and bioactive agents as described herein. In one embodiment, the entire polymer composition, and any particles made thereof, is substantially biodegradable.

In one alternative, at least one of the α-amino acids used in fabrication of the invention polymers is a biological α-amino acid. For example, when the R³s are CH₂Ph, the biological α-amino acid used in synthesis is L-phenylalanine. In alternatives wherein the R³s are CH₂—CH(CH₃)₂, the polymer contains the biological α-amino acid, L-leucine. By varying the R³s within monomers as described herein, other biological α-amino acids can also be used, e.g., glycine (when the R³s are H), alanine (when the R³s are CH₃), valine (when the R³s are CH(CH₃)₂), isoleucine (when the R³s are CH(CH₃)CH₂—CH₃), phenylalanine (when the R³s are CH₂—C₆H₅), or methionine (when the R³s are —(CH₂)₂S—CH₃), and combinations thereof. In yet another alternative embodiment, all of the various α-amino acids contained in the polymers used in making the invention therapeutic polymer compositions are biological α-amino acids, as described herein.

In yet a further embodiment wherein the polymer is a PEA, PEUR or PEU of any one of formulas (I) and (III)-(VII), at least one of the R³s further can be —(CH₂)₃—, which cyclizes to form the chemical structure described by structural formula (XIII): 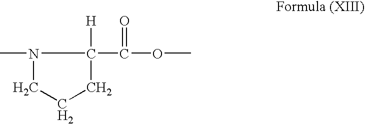
When the R³s are —(CH₂)₃—, an α-imino acid analogous to pyrrolidine-2-carboxylic acid (proline) is used.

In certain embodiments, the polymer in the invention therapeutic polymer composition plays an active role in the treatment processes at the site of local administration, e.g., by injection, by holding the polymer in an agglomeration or polymer depot at the site of injection for a period of time sufficient to allow the subject's endogenous processes to slowly release particles or polymer molecules from the agglomeration. Meanwhile, the subject's endogenous processes biodegrade the polymer backbone so as to release the incorporated therapeutic diol and/or di-acid therapeutic agents, as well as any bioactive agents dispersed in the polymer. The fragile therapeutic diols and di-acids and optional bioactive agents are protected by the more slowly biodegrading polymer to increase half-life and persistence of the therapeutic diol or di-acid and bioactive agent(s) at the site of local administration.

In addition, the polymers disclosed herein (e.g., those having structural formulas (I) and (III)-(VII), upon enzymatic degradation, provide essential amino acids and other breakdown products that can be metabolized using pathways similar to those used in metabolizing fatty acids and sugars. Uptake of the invention therapeutic polymer compositions is safe: studies have shown that the subject can metabolize/clear the polymer degradation products. These polymers and the invention therapeutic polymer compositions are, therefore, substantially non-inflammatory to the subject both at the site of injection and systemically, apart from any trauma caused by injection itself.

The PEA, PEUR and PEU polymer molecules may also have the bioactive agent attached thereto, optionally via a linker or incorporated into a crosslinker between molecules. For example, in one embodiment, the polymer is contained in a polymer-bioactive agent conjugate having structural formula VIII: 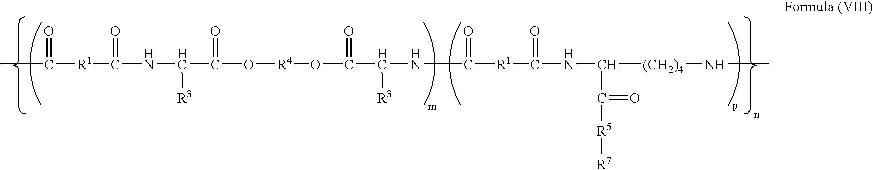
wherein n, m, p, R¹, R³, and R⁴ are as above, R⁵ is selected from the group consisting of —O—, —S—, and —NR⁸—, wherein R⁸ is H or (C₁-C₈)alkyl; and R⁷ is the bioactive agent.

In yet another embodiment, two molecules of the polymer of structural formula (IX) can be crosslinked to provide an —R⁵—R⁷—R⁵— conjugate. In another embodiment, as shown in structural formula (IX) below, the bioactive agent is covalently linked to two parts of a single polymer molecule of structural formula (III) through the —R⁵—R⁷—R⁵— conjugate and R⁵ is independently selected from the group consisting of —O—, —S—, and —NR⁸—, wherein R⁸ is H or (C₁-C₈) alkyl; and R⁷ is the bioactive agent.

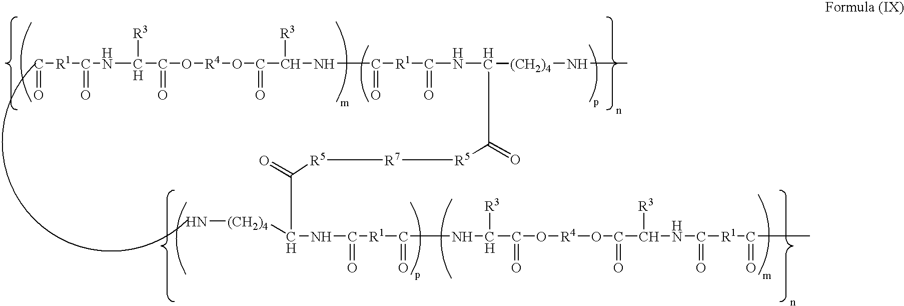

Alternatively still, as shown in structural formula (X) below, a linker, —X—Y—, can be inserted between R⁵ and bioactive agent R⁷, in the molecule of structural formula (III), wherein X is selected from the group consisting of (C₁-C₁₈) alkylene, substituted alkylene, (C₃-C₈) cycloalkylene, substituted cycloalkylene, 5-6 membered heterocyclic system containing 1-3 heteroatoms selected from the group O, N, and S, substituted heterocyclic, (C₂-C₁₈) alkenyl, substituted alkenyl, alkynyl, substituted alkynyl, C₆ and C₁₀ aryl, substituted aryl, heteroaryl, substituted heteroaryl, alkylaryl, substituted alkylaryl, arylalkynyl, substituted arylalkynyl, arylalkenyl, substituted arylalkenyl, arylalkynyl, substituted arylalkynyl and wherein the substituents are selected from the group H, F, Cl, Br, I, (C₁-C₆) alkyl, —CN, —NO₂, —OH, —O(C₁-C₄) alkyl, —S(C₁-C₆) alkyl, —S[(═O)(C₁-C₆) alkyl], —S[(O₂)(C₁-C₆) alkyl], —C[(═O)(C₁-C₆) alkyl], CF₃, —O[(CO)—(C₁-C₆) alkyl], —S(O₂)[N(R⁹R¹⁰)], —NH[(C═O)(C₁-C₆) alkyl], —NH(C═O)N(R⁹R¹⁰), —N(R⁹R¹⁰); where R⁹ and R¹⁰ are independently H or (C₁-C₆) alkyl; and Y is selected from the group consisting of —O—, —S—, —S—S—, —S(O)—, —S(O₂)—, —NR⁸—, —C(═O)—, —OC(═O)—, —C(═O)O—, —OC(═O)NH—, —NR⁸C(═O)—, —C(═O)NR⁸—, —NR⁸C(═O)NR⁸—, and —NR⁸C(═S)NR⁸—.

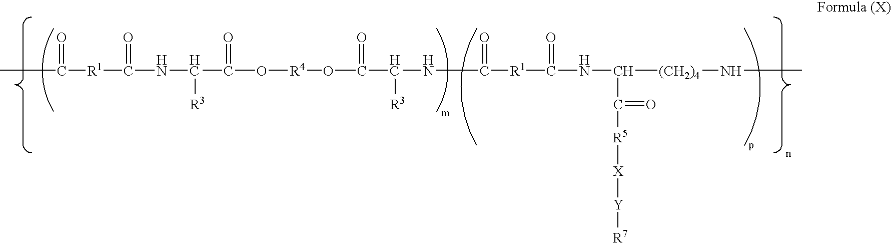

In another embodiment, two parts of a single macromolecule are covalently linked to the bioactive agent through an —R⁵—R⁷—Y—X—R⁵— bridge (Formula XI): 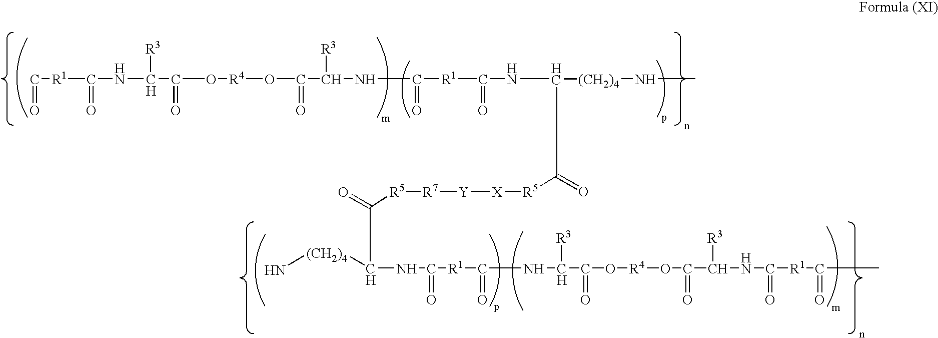
wherein, X is selected from the group consisting of (C₁-C₁₈) alkylene, substituted alkylene, (C₃-C₈) cycloalkylene, substituted cycloalkylene, 5-6 membered heterocyclic system containing 1-3 heteroatoms selected from the group O, N, and S, substituted heterocyclic, (C₂-C₁₈) alkenyl, substituted alkenyl, alkynyl, substituted alkynyl, (C₆-C₁₀) aryl, substituted aryl, heteroaryl, substituted heteroaryl, alkylaryl, substituted alkylaryl, arylalkynyl, substituted arylalkynyl, arylalkenyl, substituted arylalkenyl, arylalkynyl, substituted arylalkynyl, wherein the substituents are selected from the group consisting of H, F, Cl, Br, I, (C₁-C₆) alkyl, —CN, —NO₂, —OH, —O(C₁-C₆) alkyl, —S(C₁-C₆) alkyl, —S[(═O)(C₁-C₆) alkyl], —S[(O₂)(C₁-C₆) alkyl], —C[(═O)(C₁-C₆) alkyl], CF₃, —O[(CO)—(C₁-C₆) alkyl], —S(O₂)[N(R⁹R¹⁰)], —NH[(C═O)(C₁-C₆) alkyl], —NH(C═O)N(R⁹R¹⁰), wherein R⁹ and R¹⁰ are independently H or (C₁-C₆) alkyl, and —N(R¹¹R¹²), wherein R¹¹ and R¹² are independently selected from (C₂-C₂₀) alkylene and (C₂-C₂₀) alkenylene.

In yet another embodiment, the polymer particle delivery composition contains four molecules of the polymer, except that only two of the four molecules omit R⁷ and are crosslinked to provide a single —R⁵—X—R⁵— conjugate.

The term “aryl” is used with reference to structural formulae herein to denote a phenyl radical or an ortho-fused bicyclic carbocyclic radical having about nine to ten ring atoms in which at least one ring is aromatic. In certain embodiments, one or more of the ring atoms can be substituted with one or more of nitro, cyano, halo, trifluoromethyl, or trifluoromethoxy. Examples of aryl include, but are not limited to, phenyl, naphthyl, and nitrophenyl.

The term “alkenylene” is used with reference to structural formulae herein to mean a divalent branched or unbranched hydrocarbon chain containing at least one unsaturated bond in the main chain or in a side chain.

The molecular weights and polydispersities herein are determined by gel permeation chromatography (GPC) using polystyrene standards. More particularly, number and weight average molecular weights (Mn and Mw) are determined, for example, using a Model 510 gel permeation chromatography (Water Associates, Inc., Milford, Mass.) equipped with a high-pressure liquid chromatographic pump, a Waters 486 UV detector and a Waters 2410 differential refractive index detector. Tetrahydrofuran (THF) or N,N-dimethylacetamide (DMAc) is used as the eluent (1.0 mL/min). Polystyrene or poly(methyl methacrylate) standards having narrow molecular weight distribution were used for calibration.

Methods for making polymers containing α-amino acids in the backbone are well known in the art. For example, for the embodiment of the polymer of structural formula (I) wherein R⁴ is incorporated into an α-amino acid, for polymer synthesis the α-amino acid with pendant R³ can be converted through esterification into a bis-α,ω-diamine, for example, by condensing the α-amino acid containing pendant R³ with a diol HO—R⁴—RH. As a result, di-ester monomers with reactive α,ω-amino groups are formed. Then, the bis-α,ω-diamine is entered into a polycondensation reaction with a di-acid such as sebacic acid, or bis-activated esters, or bis-acyl chlorides, to obtain the final polymer having both ester and amide bonds (PEA). Alternatively, for example, for polymers of structure (I), instead of the di-acid, an activated di-acid derivative, e.g., bis-4-nitrophenyl diester, can be used as an activated di-acid. Additionally, a bis-di-carbonate, such as bis(4-nitrophenyl) dicarbonate, can be used as the activated species to obtain polymers containing a residue of a di-acid. In the case of PEUR polymers, a final polymer is obtained having both ester and urethane bonds.

More particularly, synthesis of the unsaturated poly(ester-amide)s (UPEAs) useful as biodegradable polymers of the structural formula (I) as disclosed above will be described, 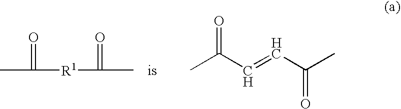
Wherein and/or (b) R⁴ is —CH₂—CH═CH—CH₂—. In cases where (a) is present and (b) is not present, R⁴ in (I) is —C₄H₈— or —C₆H₁₂—. In cases where (a) is not present and (b) is present, R¹ in (I) is —C₄H₈— or —C₈H₁₆—.

The UPEAs can be prepared by solution polycondensation of either (1) di-4-toluene sulfonic acid salt of bis(α-amino acid) di-ester of unsaturated diol and di-4-nitrophenyl ester of saturated dicarboxylic acid or (2) di-4-toluene sulfonic acid salt of bis (α-amino acid) diester of saturated diol and di-4-nitrophenyl ester of unsaturated dicarboxylic acid or (3) di-4-toluene sulfonic acid salt of bis(α-amino acid) diester of unsaturated diol and di-4-nitrophenyl ester of unsaturated dicarboxylic acid.

Salts of 4-toluene sulfonic acid are known for use in synthesizing polymers containing amino acid residues. The aryl sulfonic acid salts are used instead of the free base because the aryl sulfonic salts of bis (α-amino acid) diesters are easily purified through recrystallization and render the amino groups as unreactive ammonium tosylates throughout workup. In the polycondensation reaction, the nucleophilic amino group is readily revealed through the addition of an organic base, such as triethylamine, so the polymer product is obtained in high yield.

For polymers of structural formula (I), for example, the di-4-nitrophenyl esters of unsaturated dicarboxylic acid can be synthesized from 4-nitrophenyl and unsaturated dicarboxylic acid chloride, e.g., by dissolving triethylamine and 4-nitrophenol in acetone and adding unsaturated dicarboxylic acid chloride dropwise with stirring at −78° C. and pouring into water to precipitate product. Suitable acid chlorides included fumaric, maleic, mesaconic, citraconic, glutaconic, itaconic, ethenyl-butane dioic and 2-propenyl-butanedioic acid chlorides. For polymers of structure (IV) and (V), bis-p-nitrophenyl dicarbonates of saturated or unsaturated diols are used as the activated monomer. Dicarbonate monomers of general structure (XII) are employed for polymers of structural formula (IV) and (V), 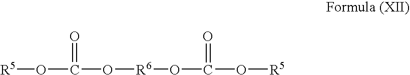
wherein each R⁵ is independently (C₆-C₁₀) aryl optionally substituted with one or more nitro, cyano, halo, trifluoromethyl, or trifluoromethoxy; and R⁶ is independently (C₂-C₂₀) alkylene, (C₂-C₂₀) alkenylene or (C₂-C₂₀) alkyloxy (C₂-C₂₀) alkenylene, fragments of 1,4:3,6-dianhydrohexitols of general formula (II), or a residue of a saturated or unsaturated therapeutic diol.

Suitable therapeutic diol compounds that can be used to prepare bis(α-amino acid) diesters of therapeutic diol monomers, or bis(carbonate) of therapeutic di-acid monomers, for introduction into the invention therapeutic polymer compositions include naturally occurring therapeutic diols, such as 17-β-estradiol, a natural and endogenous hormone, useful in preventing restenosis and tumor growth (Yang, N. N., et al. Identification of an estrogen response element activated by metabolites of 17-β-estradiol and raloxifene. Science (1996) 273, 1222-1225; Parangi, S., et al., Inhibition of angiogenesis and breast cancer in mice by the microtubule inhibitors 2-methoxyestradiol and taxol, Cancer Res. (1997) 57, 81-86; and Fotsis, T., et al., The endogenous oestrogen metabolite 2-methoxyoestradiol inhibits angiogenesis and suppresses tumor growth. Nature (1994) 368, 237-239). The safety profiles of such endogenously occurring therapeutic diol molecules are believed to be superior to those of synthetic and/or non-endogenous molecules having a similar utility, such as sirolimus.

Incorporation of a therapeutic diol into the backbone of a PEA, PEUR or PEU polymer is illustrated in this application by Example 8, in which active steroid hormone 17-β-estradiol containing mixed hydroxyls—secondary and phenolic—is introduced into the backbone of a PEA polymer. When the PEA polymer is used to fabricate particles and the particles are implanted into a patient, for example, following percutaneous transluminal coronary angioplasty (PTCA), 17-β-estradiol released from the particles in vivo can help to prevent post-implant restenosis in the patient. 17-β-estradiol, however, is only one example of a diol with therapeutic properties that can be incorporated in the backbone of a PEA, PEUR or PEU polymer in accordance with the invention. In one aspect, any bioactive steroid-diol containing primary, secondary or phenolic hydroxyls can be used for this purpose. Many steroid esters that can be made from bioactive steroid diols for use in the invention are disclosed in European application EP 0127 829 A2.

Due to the versatility of the PEA, PEUR and PEU polymers used in the invention compositions, the amount of the therapeutic diol or di-acid incorporated in the polymer backbone can be controlled by varying the proportions of the building blocks of the polymer. For example, depending on the composition of the PEA, loading of up to 40% w/w of 17-β-estradiol can be achieved. Two different regular, linear PEAs with various loading ratios of 17-β-estradiol are illustrated in Scheme 1 below:

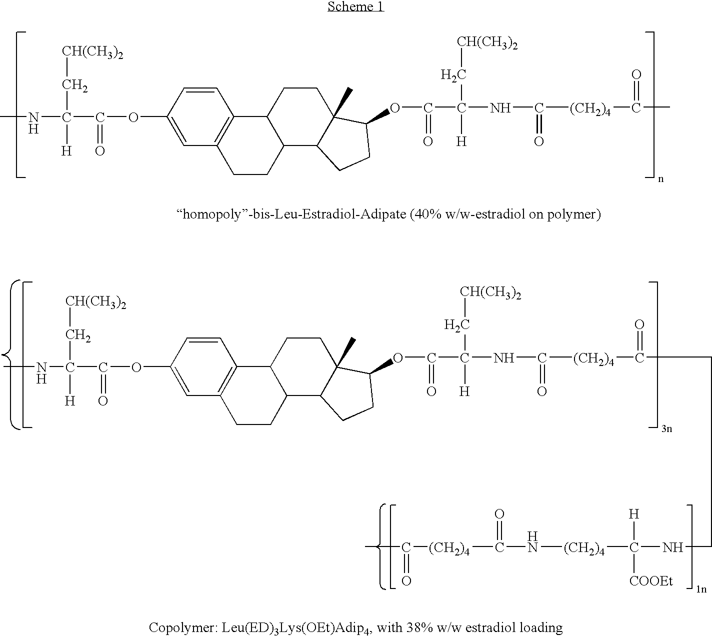

Similarly, the loading of the therapeutic diol into PEUR and PEU polymer can be varied by varying the amount of two or more building blocks of the polymer. Synthesis of a PEUR containing 17-beta-estradiol is illustrated in Example 9 below.

In addition, synthetic steroid based diols based on testosterone or cholesterol, such as 4-androstene-3, 17 diol (4-Androstenediol), 5-androstene-3, 17 diol (5-Androstenediol), 19-nor5-androstene-3, 17 diol (19-Norandrostenediol) are suitable for incorporation into the backbone of PEA and PEUR polymers according to this invention. Moreover, therapeutic diol compounds suitable for use in preparation of the invention therapeutic polymer compositions include, for example, amikacin; amphotericin B; apicycline; apramycin; arbekacin; azidamfenicol; bambermycin(s); butirosin; carbomycin; cefpiramide; chloramphenicol; chlortetracycline; clindamycin; clomocycline; demeclocycline; diathymosulfone; dibekacin, dihydrostreptomycin; dirithromycin; doxycycline; erythromycin; fortimicin(s); gentamycin(s); glucosulfone solasulfone; guamecycline; isepamicin; josamycin; kanamycin(s); leucomycin(s); lincomycin; lucensomycin; lymecycline; meclocycline; methacycline; micronomycin; midecamycin(s); minocycline; mupirocin; natamycin; neomycin; netilmicin; oleandomycin; oxytetracycline; paromycin; pipacycline; podophyllinic acid 2-ethylhydrazine; primycin; ribostamycin; rifamide; rifampin; rafamycin SV; rifapentine; rifaximin; ristocetin; rokitamycin; rolitetracycline; rasaramycin; roxithromycin; sancycline; sisomicin; spectinomycin; spiramycin; streptomycin; teicoplanin; tetracycline; thiamphenicol; theiostrepton; tobramycin; trospectomycin; tuberactinomycin; vancomycin; candicidin(s); chlorphenesin; dermostatin(s); filipin; fungichromin; kanamycin(s); leucomycins(s); lincomycin; lvcensomycin; lymecycline; meclocycline; methacycline; micronomycin; midecamycin(s); minocycline; mupirocin; natamycin; neomycin; netilmicin; oleandomycin; oxytetracycline; paramomycin; pipacycline; podophyllinic acid 2-ethylhydrazine; priycin; ribostamydin; rifamide; rifampin; rifamycin SV; rifapentine; rifaximin; ristocetin; rokitamycin; rolitetracycline; rosaramycin; roxithromycin; sancycline; sisomicin; spectinomycin; spiramycin; strepton; otbramycin; trospectomycin; tuberactinomycin; vancomycin; candicidin(s); chlorphenesin; dermostatin(s); filipin; fungichromin; meparticin; mystatin; oligomycin(s); erimycinA; tubercidin; 6-azauridine; aclacinomycin(s); ancitabine; anthramycin; azacitadine; bleomycin(s) carubicin; carzinophillin A; chlorozotocin; chromomcin(s); doxifluridine; enocitabine; epirubicin; gemcitabine; mannomustine; menogaril; atorvasi pravastatin; clarithromycin; leuproline; paclitaxel; mitobronitol; mitolactol; mopidamol; nogalamycin; olivomycin(s); peplomycin; pirarubicin; prednimustine; puromycin; ranimustine; tubercidin; vinesine; zorubicin; coumetarol; dicoumarol; ethyl biscoumacetate; ethylidine dicoumarol; iloprost; taprostene; tioclomarol; amiprilose; romurtide; sirolimus (rapamycin); tacrolimus; salicyl alcohol; bromosaligenin; ditazol; fepradinol; gentisic acid; glucamethacin; olsalazine; S-adenosylmethionine; azithromycin; salmeterol; budesonide; albuteal; indinavir; fluvastatin; streptozocin; doxorubicin; daunorubicin; plicamycin; idarubicin; pentostatin; metoxantrone; cytarabine; fludarabine phosphate; floxuridine; cladriine; capecitabien; docetaxel; etoposide; topotecan; vinblastine; teniposide, and the like. The therapeutic diol can be selected to be either a saturated or an unsaturated diol.

Suitable naturally occurring and synthetic therapeutic di-acids that can be used to prepare an amide linkage in the PEA polymer compositions of the invention include, for example, bambermycin(s); benazepril; carbenicillin; carzinophillin A; cefixime; cefininox cefpimizole; cefodizime; cefonicid; ceforanide; cefotetan; ceftazidime; ceftibuten; cephalosporin C; cilastatin; denopterin; edatrexate; enalapril; lisinopril; methotrexate; moxalactam; nifedipine; olsalazine; penicillin N; ramipril; quinacillin; quinapril; temocillin; ticarcillin; Tomudex® (N-[[5-[[(1,4-Dihydro-2-methyl-4-oxo-6-quinazolinyl)methyl]methylamino]-2-thienyl]carbonyl]-L-glutamic acid), and the like. The safety profile of naturally occurring therapeutic di-acids is believed to surpass that of synthetic therapeutic di-acids. The therapeutic di-acid can be either a saturated or an unsaturated di-acid.

The chemical and therapeutic properties of the above described therapeutic diols and di-acids as tumor inhibitors, cytotoxic antimetabolites, antibiotics, and the like, are well known in the art and detailed descriptions thereof can be found, for example, in the 13th Edition of The Merck Index (Whitehouse Station, N.J., USA).

The di-aryl sulfonic acid salts of diesters of α-amino acid and unsaturated diol can be prepared by admixing α-amino acid, e.g., 4-aryl sulfonic acid monohydrate and saturated or unsaturated diol in toluene, heating to reflux temperature, until water evolution is minimal, then cooling. The unsaturated diols include, for example, 2-butene-1,3-diol and 1,18-octadec-9-en-diol.

Saturated di-4-nitrophenyl esters of dicarboxylic acid and saturated di-4-toluene sulfonic acid salts of bis-a -amino acid esters can be prepared as described in U.S. Pat. No. 6,503,538 B1.

Synthesis of the unsaturated poly(ester-amide)s (UPEAs) useful as biodegradable polymers of the structural formula (I) as disclosed above will now be described. UPEAs having the structural formula (I) can be made in similar fashion to the compound (VII) of U.S. Pat. No. 6,503,538 B1, except that R⁴ of (III) of U.S. Pat. No. 6,503,538 and/or R¹ of (V) of U.S. Pat. No. 6,503,538 is (C₂-C₂₀) alkenylene as described above. The reaction is carried out, for example, by adding dry triethylamine to a mixture of said (III) and (IV) of U.S. Pat. No. 6,503,538 and said (V) of U.S. Pat. No. 6,503,538 in dry N,N-dimethylacetamide, at room temperature, then increasing the temperature to 60° C. and stirring for 16 hours, then cooling the reaction solution to room temperature, diluting with ethanol, pouring into water, separating polymer, washing separated polymer with water, drying to about 30° C. under reduced pressure and then purifying up to negative test on p-nitrophenol and p-toluene sulfonate. A preferred reactant (IV) of U.S. Pat. No. 6,503,538 is p-toluene sulfonic acid salt of Lysine benzyl ester, the benzyl ester protecting group is preferably removed from (II) to confer biodegradability, but it should not be removed by hydrogenolysis as in Example 22 of U.S. Pat. No. 6,503,538 because hydrogenolysis would saturate the desired double bonds; rather the benzyl ester group should be converted to an acid group by a method that would preserve unsaturation. Alternatively, the lysine reactant (IV) of U.S. Pat. No. 6,503,538 can be protected by a protecting group different from benzyl that can be readily removed in the finished product while preserving unsaturation, e.g., the lysine reactant can be protected with t-butyl (i.e., the reactant can be t-butyl ester of lysine) and the t-butyl can be converted to H while preserving unsaturation by treatment of the product (II) with acid.

A working example of the compound having structural formula (I) is provided by substituting p-toluene sulfonic acid salt of bis(L-phenylalanine) 2-butene-1,4-diester for (III) in Example 1 of U.S. Pat. No. 6,503,538 or by substituting di-p-nitrophenyl fumarate for (V) in Example 1 of U.S. Pat. No. 6,503,538 or by substituting the p-toluene sulfonic acid salt of bis(L-phenylalanine) 2-butene-1,4-diester for III in Example 1 of U.S. Pat. No. 6,503,538 and also substituting bis-p-nitrophenyl fumarate for (V) in Example 1 of U.S. Pat. No. 6,503,538.

In unsaturated compounds having either structural formula (I) or (IV), the following hold. An amino substituted aminoxyl (N-oxide) radical bearing group, e.g., 4-amino TEMPO, can be attached using carbonyldiimidazol, or suitable carbodiimide, as a condensing agent. Bioactive agents, as described herein, can be attached via the double bond functionality. Hydrophilicity can be imparted by bonding to poly(ethylene glycol) diacrylate.

In yet another aspect, the PEA and PEUR polymers contemplated for use in forming the invention therapeutic polymer compositions include those set forth in U.S. Pat. Nos. 5,516,881; 6,476,204; 6,503,538; and in U.S. application Ser. Nos. 10/096,435; 10/101,408; 10/143,572; and 10/194,965; the entire contents of each of which is incorporated herein by reference.

The biodegradable PEA, PEUR and PEU polymers can contain from one to multiple different α-amino acids per polymer molecule and preferably have weight average molecular weights ranging from 10,000 to 125,000; these polymers and copolymers typically have intrinsic viscosities at 25° C., determined by standard viscosimetric methods, ranging from 0.3 to 4.0, for example, ranging from 0.5 to 3.5.

PEA and PEUR polymers contemplated for use in the practice of the invention can be synthesized by a variety of methods well known in the art. For example, tributyltin (IV) catalysts are commonly used to form polyesters such as poly(g-caprolactone), poly(glycolide), poly(lactide), and the like. However, it is understood that a wide variety of catalysts can be used to form polymers suitable for use in the practice of the invention.

Such poly(caprolactones) contemplated for use have an exemplary structural formula (XIV) as follows:

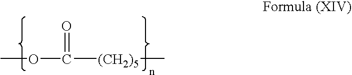

Poly(glycolides) contemplated for use have an exemplary structural formula (XV) as follows:

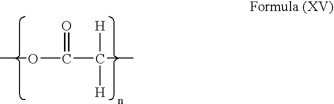

Poly(lactides) contemplated for use have an exemplary structural formula (XVI) as follows:

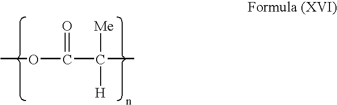

An exemplary synthesis of a suitable poly(lactide-co-ε-caprolactone) including an aminoxyl moiety is set forth as follows. The first step involves the copolymerization of lactide and ε-caprolactone in the presence of benzyl alcohol using stannous octoate as the catalyst to form a polymer of structural formula (XVII).

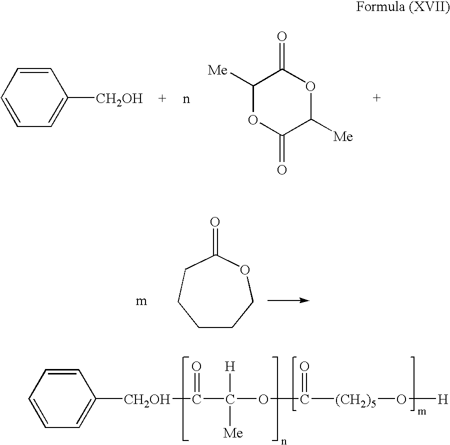

The hydroxy terminated polymer chains can then be capped with maleic anhydride to form polymer chains having structural formula (XVIII):

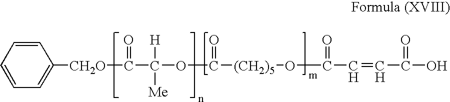

At this point, 4-amino-2,2,6,6-tetramethylpiperidine-1-oxy can be reacted with the carboxylic end group to covalently attach the aminoxyl moiety to the copolymer via the amide bond which results from the reaction between the 4-amino group and the carboxylic acid end group. Alternatively, the maleic acid capped copolymer can be grafted with polyacrylic acid to provide additional carboxylic acid moieties for subsequent attachment of further aminoxyl groups.

The description and methods of synthesis of PEA and PEUR polymers that do not have a therapeutic diol or di-acid incorporated into the backbone of the polymer are set forth in U.S. Pat. Nos. 5,516,881; 6,476,204; 6,503,538; and in U.S. application Ser. Nos. 10/096,435; 10/101,408; 10/143,572; 10/194,965; 10/362,848, 10/346,848, 10/788,747 and in provisional application No. 60/576,239, the entire content of each of which is incorporated herein by reference.

The invention bioactive PEA, PEUR and PEU polymer compositions useful in the invention methods biodegrade by enzymatic action at the surface. Therefore, the polymers, for example particles thereof, facilitate in vivo release of a bioactive agent incorporated into the backbone or dispersed in the polymer at a controlled release rate, which is specific and constant over a prolonged period. Additionally, PEA, PEUR and PEU polymers break down in vivo without production of adverse side products, the polymers in the compositions are substantially non-inflammatory.

The biodegradable PEA, PEUR and PEU polymers can contain from one to multiple different α-amino acids per polymer molecule and preferably have weight average molecular weights ranging from 10,000 to 125,000; these polymers and copolymers typically have intrinsic viscosities at 25° C., determined by standard viscosimetric methods, ranging from 0.3 to 4.0, for example, ranging from 0.5 to 3.5.

The PEU polymers disclosed herein can be fabricated as high molecular weight polymers useful for making the invention therapeutic polymer compositions for delivery to humans and other mammals of a variety of pharmaceutical and biologically active agents. The PEUs incorporate hydrolytically cleavable ester groups and non-toxic, naturally occurring monomers that contain α-amino acids in the polymer chains. The ultimate biodegradation products of PEUs will be amino acids, diols, and CO₂ In contrast to the PEAs and PEURs, the invention PEUs are crystalline or semi-crystalline and possess advantageous mechanical, chemical and biodegradation properties that allow formulation of completely synthetic, and hence easy to produce, crystalline and semi-crystalline polymer particles, for example nanoparticles. For example, the PEU polymers used in the invention therapeutic polymer compositions have high mechanical strength, and surface erosion of the PEU polymers can be catalyzed by enzymes present in physiological conditions, such as hydrolases.

In unsaturated compounds having structural formula (VII) for PEU, the following hold: An amino substituted aminoxyl (N-oxide) radical bearing group e.g., 4-amino TEMPO, can be attached using carbonyldiimidazole, or suitable carbodiimide, as a condensing agent. Bioactive agents, and the like, as described herein, optionally can be attached via the double bond functionality provided that the therapeutic diol residue in the polymer composition does not contain a double or triple bond.

For example, the invention high molecular weight semi-crystalline PEUs having structural formula (VI) can be prepared inter-facially by using phosgene as a bis-electrophilic monomer in a chloroform/water system, as shown in the reaction scheme (2) below:

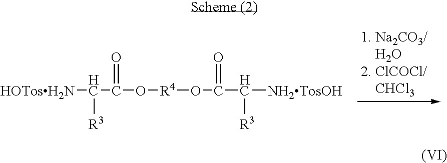

Synthesis of copoly(ester ureas) (PEUs) containing L-Lysine esters and having structural formula (VII) can be carried out by a similar Scheme (3):

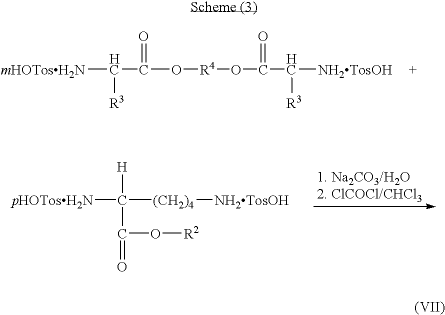

A 20% solution of phosgene (ClCOCl) (highly toxic) in toluene, for example (commercially available (Fluka Chemie, GMBH, Buchs, Switzerland), can be substituted either by diphosgene (trichloromethylchloroformate) or triphosgene (bis(trichloromethyl)carbonate). Less toxic carbonyldiimidazole can be also used as a bis-electrophilic monomer instead of phosgene, di-phosgene, or tri-phosgene.

General Procedure for Synthesis of PEUs

It is necessary to use cooled solutions of monomers to obtain PEUs of high molecular weight. For example, to a suspension of di-p-toluenesulfonic acid salt of bis(α-amino acid)-α,ω-alkylene diester in 150 mL of water, anhydrous sodium carbonate is added, stirred at room temperature for about 30 minutes and cooled to about 2-0° C., forming a first solution. In parallel, a second solution of phosgene in chloroform is cooled to about 15-10° C. The first solution is placed into a reactor for interfacial polycondensation and the second solution is quickly added at once and stirred briskly for about 15 min. Then chloroform layer can be separated, dried over anhydrous Na₂SO₄, and filtered. The obtained solution can be stored for further use.

All the exemplary PEU polymers fabricated were obtained as solutions in chloroform and these solutions are stable during storage. However, some polymers, for example, 1-Phe-4, become insoluble in chloroform after separation. To overcome this problem, polymers can be separated from chloroform solution by casting onto a smooth hydrophobic surface and allowing chloroform to evaporate to dryness. No further purification of obtained PEUs is needed. The yield and characteristics of exemplary PEUs obtained by this procedure are summarized in Table 2 herein.

General Procedure for Preparation of porous PEUs.

Methods for making the PEU polymers containing α-amino acids in the general formula will now be described. For example, for the embodiment of the polymer of formula (I) or (II), the α-amino acid can be converted into a bis(α-amino acid)-α,ω-diol-diester monomer, for example, by condensing the ═-amino acid with a diol HO—R¹—OH. As a result, ester bonds are formed. Then, acid chloride of carbonic acid (phosgene, diphosgene, triphosgene) is entered into a polycondensation reaction with, a di-p-toluenesulfonic acid salt of a bis(α-amino acid)-alkylene diester to obtain the final polymer having both ester and urea bonds. In the present invention, at least one therapeutic diol can be used in the polycondensation protocol.

The unsaturated PEUs can be prepared by interfacial solution condensation of di-p-toluenesulfonate salts of bis(α-amino acid)-alkylene diesters, comprising at least one double bond in R¹. Unsaturated diols useful for this purpose include, for example, 2-butene-1,4-diol and 1,18-octadec-9-en-diol. Unsaturated monomer can be dissolved prior to the reaction in alkaline water solution, e.g. sodium hydroxide solution. The water solution can then be agitated intensely, under external cooling, with an organic solvent layer, for example chloroform, which contains an equimolar amount of monomeric, dimeric or trimeric phosgene. An exothermic reaction proceeds rapidly, and yields a polymer that (in most cases) remains dissolved in the organic solvent. The organic layer can be washed several times with water, dried with anhydrous sodium sulfate, filtered, and evaporated. Unsaturated PEUs with a yield of about 75%-85% can be dried in vacuum, for example at about 45° C.

To obtain a porous, strong material, L-Leu based PEUs, such as 1-L-Leu-4 and 1-L-Leu-6, can be fabricated using the general procedure described below. Such procedure is less successful in formation of a porous, strong material when applied to L-Phe based PEUs.

The reaction solution or emulsion (about 100 mL) of PEU in chloroform, as obtained just after interfacial polycondensation, is added dropwise with stirring to 1,000 mL of about 80° C.-85° C. water in a glass beaker, preferably a beaker made hydrophobic with dimethyldichlorsilane to reduce the adhesion of PEU to the beaker's walls. The polymer solution is broken in water into small drops and chloroform evaporates rather vigorously. Gradually, as chloroform is evaporated, small drops combine into a compact tar-like mass that is transformed into a sticky rubbery product. This rubbery product is removed from the beaker and put into hydrophobized cylindrical glass-test-tube, which is thermostatically controlled at about 80° C. for about 24 hours. Then the test-tube is removed from the thermostat, cooled to room temperature, and broken to obtain the polymer. The obtained porous bar is placed into a vacuum drier and dried under reduced pressure at about 80° C. for about 24 hours. In addition, any procedure known in the art for obtaining porous polymeric materials can also be used.

Properties of high-molecular-weight porous PEUs made by the above procedure yielded results as summarized in Table 2.

TABLE 2
Properties of PEU Polymers of Formula (VI) and (VII)

| | | η_{red}^{a)} | | | | Tg^{c)} | Tₘ^{c)} |
|---|---|---|---|---|---|---|---|
| PEU* | Yield [%] | [dL/g] | M_{w}^{b)} | Mₙ^{b)} | M_{w}/Mₙ^{b)} | [° C.] | [° C.] |
| 1-L-Leu-4 | 80 | 0.49 | 84000 | 45000 | 1.90 | 67 | 103 |
| 1-L-Leu-6 | 82 | 0.59 | 96700 | 50000 | 1.90 | 64 | 126 |
| 1-L-Phe-6 | 77 | 0.43 | 60400 | 34500 | 1.75 | — | 167 |
| [1-L-Leu-6]_{0.75}-[1-L- | 84 | 0.31 | 64400 | 43000 | 1.47 | 34 | 114 |
| Lys(OBn)]_{0.25} | | | | | | | |
| 1-L-Leu-DAS | 57 | 0.28 | 55700^{d)} | 27700^{d)} | 2.1^{d)} | 56 | 165 |

*PEUs of general formula (VI), where, 1-L-Leu-4: R⁴ = (CH₂)₄, R³ = i-C₄H₉ 1-L-Leu-6: R⁴ = (CH₂)₆, R³ = i-C₄H₉ 1-L-Phe-6: R⁴ = (CH₂)₆, R³ = —CH₂—C₆H₅. 1-L-Leu-DAS: R⁴ = 1,4:3,6-dianhydrosorbitol, R³ = i-C₄H
^{a)}Reduced viscosities were measured in DMF at 25° C. and a concentration 0.5 g/dL.
^{b)}GPC Measurements were carried out in DMF, (PMMA).
^{c)}Tg taken from second heating curve from DSC Measurements (heating rate 10° C./min).
^{d)}GPC Measurements were carried out in DMAc, (PS).

Tensile strength of illustrative synthesized PEUs was measured and results are summarized in Table 3. Tensile strength measurement was obtained using dumbbell-shaped PEU films (4×1.6 cm), which were cast from chloroform solution with average thickness of 0.125 mm and subjected to tensile testing on tensile strength machine (Chatillon TDC200) integrated with a PC using Nexygen FM software (Amtek, Largo, Fla.) at a crosshead speed of 60 mm/min. Examples illustrated herein can be expected to have the following mechanical properties:

1. A glass transition temperature in the range from about 30° C. to about 90° C., for example, in the range from about 35° C. to about 70° C.;

2. A film of the polymer with average thickness of about 1.6 cm will have tensile stress at yield of about 20 Mpa to about 150 Mpa, for example, about 25 Mpa to about 60 Mpa;

3. A film of the polymer with average thickness of about 1.6 cm will have a percent elongation of about 10% to about 200%, for example about 50% to about 150%; and

4. A film of the polymer with average thickness of about 1.6 cm will have a Young's modulus in the range from about 500 MPa to about 2000 MPa. Table 2 below summarizes the properties of exemplary PEUs of this type.

TABLE 3
Mechanical Properties of PEUs

| | | Tensile Stress | | Young's |
|---|---|---|---|---|
| | Tg^{a)} | at Yield | Percent | Modulus |
| Polymer designation | (° C.) | (MPa) | Elongation (%) | (MPa) |
| 1-L-Leu-6 | 64 | 21 | 114 | 622 |
| [1-L-Leu-6]_{0.75}- | 34 | 25 | 159 | 915 |
| [1-L-Lys(OBn)]_{0.25} | | | | |

The PEA, PEUR and PEU polymers described herein can be fabricated in a variety of molecular weights and a variety of w/w% concentrations of the therapeutic diol or di-acid in the backbone of the polymer. The appropriate molecular weight for use with a given concentration of bioactive agent is readily determined by one of skill in the art. Thus, e.g., a suitable molecular weight will be on the order of about 5,000 to about 300,000, for example about 5,000 to about 250,000, or about 75,000 to about 200,000, or about 100,000 to about 150,000 and a suitable w/w% concentration of a residue of a bioactive agent incorporated into the backbone of the polymer will be on the order of about 5 w/w % to about 70 w/w %, for example about 10 w/w % to about 40 w/w %, or about 20 w/w % to about 40 w/w %. The amount of bioactive agent incorporated into the backbone of the polymer will be highest in the case of a homopolymer (e.g., containing no Lysine-based monomer) that incorporates both a therapeutic diol and a therapeutic di-acid.

The molecular weights and polydispersities herein are determined by gel permeation chromatography (GPC) using polystyrene standards. More particularly, number and weight average molecular weights (Mₙ and M_{w}) are determined, for example, using a Model 510 gel permeation chromatography (Water Associates, Inc., Milford, Mass.) equipped with a high-pressure liquid chromatographic pump, a Waters 486 UV detector and a Waters 2410 differential refractive index detector. Tetrahydrofuran (THF) or N,N-dimethylacetamide (DMAc) is used as the eluent (1.0 mL/min). The polystyrene standards have a narrow molecular weight distribution.

While the optional bioactive agent(s) can be dispersed within the polymer matrix without chemical linkage to the polymer carrier, it is also contemplated that one or more bioactive agents or covering molecules can be covalently bound to the biodegradable polymers via a wide variety of suitable functional groups. For example, a free carboxyl group can be used to react with a complimentary moiety on a bioactive agent or covering molecule, such as an hydroxy, amino, or thio group, and the like. A wide variety of suitable reagents and reaction conditions are disclosed, e.g., in March's Advanced Organic Chemistry, Reactions, Mechanisms, and Structure, Fifth Edition, (2001); and Comprehensive Organic Transformations, Second Edition, Larock (1999).

In other embodiments, one or more bioactive agent can be linked to any of the polymers of structures (I) and (III-VII) through an amide, ester, ether, amino, ketone, thioether, sulfinyl, sulfonyl, or disulfide linkage. Such a linkage can be formed from suitably functionalized starting materials using synthetic procedures that are known in the art.

For example, in one embodiment a polymer can be linked to a bioactive agent via a free carboxyl group (e.g., COOH) of the polymer. Specifically, a compound of structures (I) and (III) can react with an amino functional group or a hydroxyl functional group of a bioactive agent to provide a biodegradable polymer having the bioactive agent attached via an amide linkage or ester linkage, respectively. In another embodiment, the carboxyl group of the polymer can be benzylated or transformed into an acyl halide, acyl anhydride/“mixed” anhydride, or active ester. In other embodiments, the free —NH₂ ends of the polymer molecule can be acylated to assure that the bioactive agent will attach only via a carboxyl group of the polymer and not to the free ends of the polymer.

Water soluble covering molecule(s), such as poly(ethylene glycol) (PEG); phosphatidylcholine (PC); glycosaminoglycans including heparin; polysaccharides including chitosan, alginates and polysialic acid; poly(ionizable or polar amino acids) including polyserine, polyglutamic acid, polyaspartic acid, polylysine and polyarginine; as described herein, and targeting molecules, such as antibodies, antigens and ligands, are bioactive agents that can also be conjugated to the polymer on the exterior of particles formed from the therapeutic polymer composition after production of the particles to block active sites not occupied by a bioactive agent or to target delivery of the particles to a specific body site as is known in the art. The molecular weights of PEG molecules on a single particle can be substantially any molecular weight in the range from about 200 to about 200,000, so that the molecular weights of the various PEG molecules attached to the particle can be varied.

Alternatively, a bioactive agent or covering molecule can be attached to the polymer via a linker molecule or by cross-linking two or more molecules of the polymer as described herein. Indeed, to improve surface hydrophobicity of the biodegradable polymer, to improve accessibility of the biodegradable polymer towards enzyme activation, and to improve the release profile of the bioactive agents from the biodegradable polymer, a linker may be utilized to indirectly attach a bioactive agent to the biodegradable polymer. In certain embodiments, the linker compounds include poly(ethylene glycol) having a molecular weight (MW) of about 44 to about 10,000, preferably 44 to 2000; amino acids, such as serine; polypeptides with repeat number from 1 to 100; and any other suitable low molecular weight polymers. The linker typically separates the bioactive agent from the polymer by about 5 angstroms up to about 200 angstroms.

In still further embodiments, the linker is a divalent radical of formula W-A-Q, wherein A is (C₁-C₂₄) alkyl, (C₂-C₂₄) alkenyl, (C₂-C₂₄) alkynyl, (C₂-C₂₀) alkyloxy, (C₃-C₈) cycloalkyl, or (C₆-C₁₀) aryl, and W and Q are each independently —N(R)C(═O)—, —C(═O)N(R)—, —OC(═O)—, —C(═O)O, —O—, —S—, —S(O), —S(O)₂—, —S—S—, —N(R)—, —C(═O)—, wherein each R is independently H or (C₁-C₆) alkyl.

As used to describe the above linkers, the term “alkyl” refers to a straight or branched chain hydrocarbon group including methyl, ethyl, n-propyl, isopropyl, n-butyl, isobutyl, tert-butyl, n-hexyl, and the like.

As used herein used to describe the above linkers, “alkenyl” refers to straight or branched chain hydrocarbyl groups having one or more carbon-carbon double bonds.

As used herein used to describe the above linkers, “alkynyl” refers to straight or branched chain hydrocarbyl groups having at least one carbon-carbon triple bond.

As used herein used to describe the above linkers, “aryl” refers to aromatic groups having in the range of 6 up to 14 carbon atoms.

In certain embodiments, the linker may be a polypeptide having from about 2 up to about 25 amino acids. Suitable peptides contemplated for use include poly-L-glycine, poly-L-lysine, poly-L-glutamic acid, poly-L-aspartic acid, poly-L-histidine, poly-L-omithine, poly-L-serine, poly-L-threonine, poly-L-tyrosine, poly-L-leucine, poly-L-lysine-L-phenylalanine, poly-L-arginine, poly-L-lysine-L-tyrosine, and the like.

In one embodiment, a bioactive agent can covalently crosslink the polymer, i.e. the bioactive agent is bound to more than one polymer molecule, to form an intermolecular bridge. This covalent crosslinking can be done with or without a linker containing a bioactive agent.

A bioactive agent molecule can also be incorporated into an intramolecular bridge by covalent attachment between two sites on the same polymer molecule.

A linear polymer polypeptide conjugate is made by protecting the potential nucleophiles on the polypeptide backbone and leaving only one reactive group to be bound to the polymer or polymer linker construct. Deprotection is performed according to methods well known in the art for deprotection of peptides (Boc and Fmoc chemistry for example).

In one embodiment of the present invention, a bioactive agent is a polypeptide presented as a retro-inverso or partial retro-inverso peptide.

In other embodiments, a bioactive agent may be mixed with a photocrosslinkable version of the polymer in a matrix, and, after crosslinking, the material is dispersed (ground) to form particles having an average diameter in the range from about 0.1 to about 1 μm.

The linker can be attached first to the polymer or to the bioactive agent or covering molecule. During synthesis, the linker can be either in unprotected form or protected from, using a variety of protecting groups well known to those skilled in the art. In the case of a protected linker, the unprotected end of the linker can first be attached to the polymer or the bioactive agent or covering molecule. The protecting group can then be de-protected using Pd/H₂ hydrogenation for saturated polymer backbones, mild acid or base hydrolysis for unsaturated polymers, or any other common de-protection method that is known in the art. The de-protected linker can then be attached to the bioactive agent or covering molecule, or to the polymer

An exemplary conjugate synthesis performed on a biodegradable polymer according to the invention (wherein the molecule to be attached to the polymer is an amino substituted aminoxyl N-oxide radical) is set forth as follows. A biodegradable polymer herein can be reacted with an aminoxyl radical containing compound, e.g., 4-amino-2,2,6,6-tetramethylpiperidine-1-oxy, in the presence of N,N′-carbonyl diimidazole or suitable carbodiimide, to replace the hydroxyl moiety in the carboxyl group, either on the pendant carboxylic acids of the PEAs, PEURs or PEUs, or at the chain end of a polyester as described, with an amide linkage to the aminoxyl (N-oxide) radical containing group. The amino moiety covalently bonds to the carbon of the carbonyl residue such that an amide bond is formed. The N,N′-carbonyldiimidazole or suitable carbodiimide converts the hydroxyl moiety in the carboxyl group at the chain end of the polyester into an intermediate activated moiety which will react with the amino group of the aminoxyl (N oxide) radical compound, e.g., the amine at position 4 of 4-amino-2,2,6,6-tetramethylpiperidine-1-oxy. The aminoxyl reactant is typically used in a mole ratio of reactant to polyester ranging from 1:1 to 100:1. The mole ratio of N,N′-carbonyldiimidazole or carbodiimide to aminoxyl is preferably about 1:1.

A typical reaction is as follows. A polyester is dissolved in a reaction solvent and reaction is readily carried out at the temperature utilized for the dissolving. The reaction solvent may be any in which the polyester will dissolve; this information is normally available from the manufacturer of the polyester. When the polyester is a polyglycolic acid or a poly(glycolide-L-lactide) (having a monomer mole ratio of glycolic acid to L-lactic acid greater than 50:50), highly refined (99.9+% pure) dimethyl sulfoxide at 115° C. to 130° C. or DMSO at room temperature suitably dissolves the polyester. When the polyester is a poly-L-lactic acid, a poly-DL-lactic acid or a poly(glycolide-L-lactide) (having a monomer mole ratio of glycolic acid to L-lactic acid 50:50 or less than 50:50), tetrahydrofuran, dichloromethane (DCM) and chloroform at room temperature to 40˜50° C. suitably dissolve the polyester.

The product may be precipitated from the reaction mixture by adding cold non-solvent for the product. For example, aminoxyl-containing polyglycolic acid and aminoxyl-containing poly(glycolide-L-lactide) formed from glycolic acid-rich monomer mixture are readily precipitated from hot dimethylsulfoxide by adding cold methanol or cold acetone/methanol mixture and then recovered, e.g., by filtering. When the product is not readily precipitated by adding cold non-solvent for the product, the product and solvent may be separated by using vacuum techniques. For example, aminoxyl-containing poly-L-lactic acid is advantageously separated from solvent in this way. The recovered product is readily further purified by washing away water and by-products (e.g. urea) with a solvent which does not dissolve the product, e.g., methanol in the case of the modified polyglycolic acid, polylactic acid and poly(glycolide-L-lactide) products herein. Residual solvent from such washing may be removed using vacuum drying.

Polymer—Bioactive agent Linkage

In one embodiment, the polymers used to make the invention therapeutic polymer compositions as described herein have one or more bioactive agent directly linked to the polymer. The residues of the polymer can be linked to the residues of the one or more bioactive agents. For example, one residue of the polymer can be directly linked to one residue of a bioactive agent. The polymer and the bioactive agent can each have one open valence. Alternatively, more than one bioactive agent, multiple bioactive agents, or a mixture of bioactive agents having different therapeutic or palliative activity can be directly linked to the polymer. However, since the residue of each bioactive agent can be linked to a corresponding residue of the polymer, the number of residues of the one or more bioactive agents can correspond to the number of open valences on the residue of the polymer having at least one diol or di-acid bioactive agent incorporated into the backbone of the polymer.

As used herein, a “residue of a polymer” refers to a radical of a polymer having one or more open valences. Any synthetically feasible atom, atoms, or functional group of the polymer (e.g., on the polymer backbone or pendant group) is substantially retained when the radical is attached to a residue of a bioactive agent. Additionally, any synthetically feasible functional group (e.g., carboxyl) can be created on the polymer (e.g., on the polymer backbone as a pendant group or as chain termini) to provide the open valence, provided bioactivity of the backbone therapeutic agent is substantially retained when the radical is attached to a residue of a bioactive agent. Based on the linkage that is desired, those skilled in the art can select suitably functionalized starting materials that can be used to derivatize the PEA and PEUR polymers used in the present invention using procedures that are known in the art.

As used herein, a “residue of a compound of structural formula (*)” refers to a radical of a compound of polymer formulas (I), (III)-(VII) as described herein having one or more open valences. Any synthetically feasible atom, atoms, or functional group of the compound (e.g., on the polymer backbone, pendant or end group) can be removed to provide the open valence, provided bioactivity of the backbone therapeutic agent is substantially retained when the radical is attached. Additionally, any synthetically feasible functional group (e.g., carboxyl) can be created on the compound of formulas (I), (III)-(VII) (e.g., on the polymer backbone or pendant group) to provide the open valence, provided bioactivity of the backbone therapeutic agent is substantially retained when the radical is attached to a residue of a bioactive agent. Based on the linkage that is desired, those skilled in the art can select suitably functionalized starting materials that can be used to derivatize the compound of formulas (I), (III)-(VII) using procedures that are known in the art.

For example, the residue of a bioactive agent can be linked to the residue of a compound of structural formula (I)-(III)-(VII) through an amide (e.g., —N(R)C(═O)— or —C(═O)N(R)—), ester (e.g., —OC(═O)— or —C(═O)O—), ether (e.g., —O—), amino (e.g., —N(R)—), ketone (e.g., —C(═O)—), thioether (e.g., —S—), sulfinyl (e.g., —S(O)—), sulfonyl (e.g., —S(O)₂—), disulfide (e.g., —S—S—), or a direct (e.g., C—C bond) linkage, wherein each R is independently H or (C₁-C₆) alkyl. Such a linkage can be formed from suitably functionalized starting materials using synthetic procedures that are known in the art. Based on the linkage that is desired, those skilled in the art can select suitably functional starting material to derivatize any residue of a compound of structural formula (I) or (III)-(VII) and thereby conjugate a given residue of a bioactive agent using procedures that are known in the art. The residue of the optional bioactive agent can be linked to any synthetically feasible position on the residue of a compound of structural formula (I) or (III)-(VII). Additionally, the invention also provides compounds having more than one residue of a bioactive agent directly linked to a compound of structural formula (I), (III)-(VII).

The number of bioactive agents that can be linked to the polymer molecule can typically depend upon the molecular weight of the polymer and the number of backbone therapeutic agents incorporated into the polymer. For example, for a compound of structural formula (I), wherein n is about 5 to about 150, preferably about 5 to about 70, up to about 150 bioactive agent molecules (i.e., residues thereof) can be directly linked to the polymer (i.e., residue thereof) by reacting the bioactive agent with backbone, pendant or terminal groups of the polymer. The number of sites for linkage of a bioactive agent in the invention therapeutic polymer compositions is accordingly reduced by the number of backbone therapeutic diol or di-acids incorporated into the polymer. In unsaturated polymers, bioactive agents can also be reacted with double (or triple) bonds in the polymer, provided that the therapeutic diol or di-acid residues incorporated into the polymer backbone do not contain any double (or triple) bonds themselves. Hence, in the case of estradiol incorporated into the backbone, linkage of a bioactive agent at a double bond in the polymer composition would not be recommended, to prevent bonding of the bioactive agent to a double bond in the backbone diol or di-acid residue (i.e., the estradiol) in a reaction.

In the therapeutic polymer composition, either in the form of particles or not, a bioactive agent can be covalently attached directly to the polymer, rather than being dispersed by “loading” into the polymer without chemical attachment, using any of several methods well known in the art and as described hereinbelow. The amount of bioactive agent is generally approximately 0.1% to about 60% (w/w) bioactive agent to polymer composition, more preferably about 1% to about 25% (w/w) bioactive agent, and even more preferably about 2% to about 20% (w/w) bioactive agent. The percentage of bioactive agent will depend on the desired dose and the condition being treated, as discussed in more detail below.

In addition to serving as stand-alone delivery systems for therapeutic diols and di-acids (and optional bioactive agents) when directly administered in vivo, for example, in the form of inhalants, implants or local or systemic injectables, the invention therapeutic polymer compositions can be used in the fabrication of polymer coatings for various types of surgical devices. In this embodiment, the polymer coating on the surgical device is effective for controlled delivery to surrounding tissue of the therapeutic diol or di-acid as well as any bioactive agents dispersed in the polymer or covalently attached to the surface of a particle thereof.

In one embodiment, the invention therapeutic polymer composition can be fabricated in the form of a pad, sheet or wrap of any desired surface area. For example, the polymer can be woven or formed as a thin sheet of randomly oriented fibers. Such pads, sheets and wraps can be used in a number of types of wound dressings for treatment of a variety of conditions, for example by promoting endogenous healing processes at a wound site. The polymer compositions in the wound dressing biodegrade over time, releasing the therapeutic diol or di-acid to be absorbed into a target cell in a wound site where it acts intracellularly, either within the cytosol, the nucleus, or both, or the bioactive agent can bind to a cell surface receptor molecule to elicit a cellular response without entering the cell. Alternatively, the therapeutic diol or di-acid released from the polymer composition, for example when used as the covering for a bioactive stent, promotes endogenous healing processes at the wound site by contact with the surroundings into which the wound dressing or implant is placed. A detailed description of wound dressings, wound healing implants and surgical device coatings made using PEA and PEUR polymers is found in co-pending U.S. patent application Ser. No. 11/128,903, filed May 12, 2005.

A detailed description of methods of making polymer particles using PEA and PEUR polymers may be found in co-pending U.S. provisional application Nos. 60/654,715, filed Feb. 17, 2005, and 60/674,670, May 25, 2005, each of which is incorporated herein in its entirety.

Bioactive agents contemplated for dispersion within the polymers used in the invention therapeutic polymer compositions include anti-proliferants, rapamycin and any of its analogs or derivatives, paclitaxel or any of its taxene analogs or derivatives, everolimus, sirolimus, tacrolimus, or any of its -limus named family of drugs, and statins such as simvastatin, atorvastatin, fluvastatin, pravastatin, lovastatin, rosuvastatin, geldanamycins, such as 17AAG (17-allylamino-17-demethoxygeldanamycin); Epothilone D and other epothilones, 17-dimethylaminoethylamino-17-demethoxy-geldanamycin and other polyketide inhibitors of heat shock protein 90 (Hsp90), cilostazol, and the like.

Suitable bioactive agents for dispersion in the invention therapeutic polymer compositions and particles made therefrom also can be selected from those that promote endogenous production of a therapeutic natural wound healing agent, such as nitric oxide, which is endogenously produced by endothelial cells. Alternatively the bioactive agents released from the polymers during degradation may be directly active in promoting natural wound healing processes by endothelial cells. These bioactive agents can be any agent that donates, transfers, or releases nitric oxide, elevates endogenous levels of nitric oxide, stimulates endogenous synthesis of nitric oxide, or serves as a substrate for nitric oxide synthase or that inhibits proliferation of smooth muscle cells. Such agents include, for example, aminoxyls, furoxans, nitrosothiols, nitrates and anthocyanins; nucleosides such as adenosine and nucleotides such as adenosine diphosphate (ADP) and adenosine triphosphate (ATP); neurotransmitter/neuromodulators such as acetylcholine and 5-hydroxytryptamine (serotonin/5-HT); histamine and catecholamines such as adrenalin and noradrenalin; lipid molecules such as sphingosine-1-phosphate and lysophosphatidic acid; amino acids such as arginine and lysine; peptides such as the bradykinins, substance P and calcium gene-related peptide (CGRP), and proteins such as insulin, vascular endothelial growth factor (VEGF), and thrombin.

A variety of bioactive agents, coating molecules and ligands for bioactive agents can be attached, for example covalently, to the surface of the polymer coatings or particles. Bioactive agents, such as targeting antibodies, polypeptides (e.g., antigens) and drugs can be covalently conjugated to the surface of the polymer coatings or particles. In addition, coating molecules, such as polyethylene glycol (PEG) as a ligand for attachment of antibodies or polypeptides or phosphatidylcholine (PC) as a means of blocking attachment sites on the surface of the particles, can be surface-conjugated to the particles to prevent the particles from sticking to non-target biological molecules and surfaces in a subject to which the particles are administered.

For example, small proteinaceous motifs, such as the B domain of bacterial Protein A and the functionally equivalent region of Protein G are known to bind to, and thereby capture, antibody molecules by the Fc region. Such proteinaceous motifs can be attached as bioactive agents to the invention therapeutic polymer compositions, especially to the surface of the polymer particles described herein. Such molecules will act, for example, as ligands to attach antibodies for use as targeting ligands or to capture antibodies to hold precursor cells or capture cells out of the blood stream. Therefore, the antibody types that can be attached to polymer coatings using a Protein A or Protein G functional region are those that contain an Fc region. The capture antibodies will in turn bind to and hold precursor cells, such as progenitor cells, near the polymer surface while the precursor cells, which are preferably bathed in a growth medium within the polymer, secrete various factors and interact with other cells of the subject. In addition, one or more bioactive agents dispersed in the polymer particles, such as the bradykinins, may activate the precursor cells.

In addition, bioactive agents for attaching precursor cells or for capturing progenitor endothelial cells (PECs) from a blood stream in a subject to which the polymer compositions are administered are monoclonal antibodies directed against a known precursor cell surface marker. For example, complementary determinants (CDs) that have been reported to decorate the surface of endothelial cells include CD31, CD34, CD102, CD105, CD106, CD109, CDw130, CD141, CD142, CD143, CD144, CDw145, CD146, CD147, and CD166. These cell surface markers can be of varying specificity and the degree of specificity for a particular cell/developmental type/stage is in many cases not fully characterized. In addition, these cell marker molecules against which antibodies have been raised will overlap (in terms of antibody recognition) especially with CDs on cells of the same lineage: monocytes in the case of endothelial cells. Circulating endothelial progenitor cells are some way along the developmental pathway from (bone marrow) monocytes to mature endothelial cells. CDs 106, 142 and 144 have been reported to mark mature endothelial cells with some specificity. CD34 is presently known to be specific for progenitor endothelial cells and therefore is currently preferred for capturing progenitor endothelial cells out of blood in the site into which the polymer particles are implanted for local delivery of the active agents. Examples of such antibodies include single-chain antibodies, chimeric antibodies, monoclonal antibodies, polyclonal antibodies, antibody fragments, Fab fragments, IgA, IgG, IgM, IgD, IgE and humanized antibodies, and active fragments thereof.

The following bioactive agents and small molecule drugs will be particularly effective for dispersion within the invention therapeutic polymer compositions, whether sized to form a time release biodegradable polymer depot for local delivery of the bioactive agents, or sized for entry into systemic circulation, as described herein. The bioactive agents that are dispersed in the invention therapeutic polymer compositions and methods of use will be selected for their suitable therapeutic or palliative effect in treatment of a disease of interest, or symptoms thereof, or in experiments designed for in vitro testing of such effects in cells or tissue culture, or in vivo.

In one embodiment, the suitable bioactive agents are not limited to, but include, various classes of compounds that facilitate or contribute to wound healing when presented in a time-release fashion. Such bioactive agents include wound-healing cells, including certain precursor cells, which can be protected and delivered by the biodegradable polymer in the invention compositions. Such wound healing cells include, for example, pericytes and endothelial cells, as well as inflammatory healing cells. To recruit such cells to the site of a polymer depot in vivo, the invention therapeutic polymer compositions and particles thereof used in the invention and methods of use can include ligands for such cells, such as antibodies and smaller molecule ligands, that specifically bind to “cellular adhesion molecules” (CAMs). Exemplary ligands for wound healing cells include those that specifically bind to Intercellular adhesion molecules (ICAMs), such as ICAM-1 (CD54 antigen); ICAM-2 (CD102 antigen); ICAM-3 (CD50 antigen); ICAM-4 (CD242 antigen); and ICAM-5; Vascular cell adhesion molecules (VCAMs), such as VCAM-1 (CD106 antigen); Neural cell adhesion molecules (NCAMs), such as NCAM-1 (CD56 antigen); or NCAM-2; Platelet endothelial cell adhesion molecules PECAMs, such as PECAM-1 (CD31 antigen); Leukocyte-endothelial cell adhesion molecules (ELAMs), such as LECAM-1; or LECAM-2 (CD62E antigen), and the like.

In another aspect, the suitable bioactive agents include extra cellular matrix proteins, macromolecules that can be dispersed into the polymer particles used in the invention therapeutic polymer compositions, e.g., attached either covalently or non-covalently. Examples of useful extra-cellular matrix proteins include, for example, glycosaminoglycans, usually linked to proteins (proteoglycans), and fibrous proteins (e.g., collagen; elastin; fibronectins and laminin). Bio-mimics of extra-cellular proteins can also be used. These are usually non-human, but biocompatible, glycoproteins, such as alginates and chitin derivatives. Wound healing peptides that are specific fragments of such extra-cellular matrix proteins and/or their bio-mimics can also be used.

Proteinaceous growth factors are another category of bioactive agents suitable for dispersion in the invention therapeutic polymer compositions and methods of use described herein. Such bioactive agents are effective in promoting wound healing and other disease states as is known in the art, for example, Platelet Derived Growth Factor-BB (PDGF-BB), Tumor Necrosis Factor-α (TNF-α), Epidermal Growth Factor (EGF), Keratinocyte Growth Factor (KGF), Thymosin B4; and, various angiogenic factors such as vascular Endothelial Growth Factors (VEGFs), Fibroblast Growth Factors (FGFs), Tumor Necrosis Factor-beta (TNF-beta), and Insulin-like Growth Factor-1 (IGF-1). Many of these proteinaceous growth factors are available commercially or can be produced recombinantly using techniques well known in the art.

Alternatively, expression systems comprising vectors, particularly adenovirus vectors, incorporating genes encoding a variety of biomolecules can be dispersed in the invention therapeutic polymer compositions and particles thereof for timed release delivery. Methods of preparing such expression systems and vectors are well known in the art. For example, proteinaceous growth factors can be dispersed into the invention therapeutic polymer compositions for administration of the growth factors either to a desired body site for local delivery, by selection of particles sized to form a polymer depot, or systemically, by selection of particles of a size that will enter the circulation. Growth factors, such as VEGFs, PDGFs, FGF, NGF, and evolutionary and functionally related biologics, and angiogenic enzymes, such as thrombin, may also be used as bioactive agents in the invention compositions.

Small molecule drugs are yet another category of bioactive agents suitable for dispersion in the invention therapeutic polymer compositions and methods of use described herein. Such drugs include, for example, antimicrobials and anti-inflammatory agents as well as certain healing promoters, such as, for example, vitamin A and synthetic inhibitors of lipid peroxidation.

A variety of antibiotics can be dispersed as bioactive agents in the invention therapeutic polymer compositions to indirectly promote natural healing processes by preventing or controlling infection. Suitable antibiotics include many classes, such as aminoglycoside antibiotics or quinolones or beta-lactams, such as cefalosporins, e.g., ciprofloxacin, gentamycin, tobramycin, erythromycin, vancomycin, oxacillin, cloxacillin, methicillin, lincomycin, ampicillin, and colistin. Suitable antibiotics have been described in the literature.

Suitable antimicrobials include, for example, Adriamycin PFS/RDF® (Pharmacia and Upjohn), Blenoxane® (Bristol-Myers Squibb Oncology/Immunology), Cerubidine® (Bedford), Cosmegen® (Merck), DaunoXome® (NeXstar), Doxil® (Sequus), Doxorubicin Hydrochloride® (Astra), Idamycin® PFS (Pharmacia and Upjohn), Mithracin® (Bayer), Mitamycin® (Bristol-Myers Squibb Oncology/Immunology), Nipen® (SuperGen), Novantrone® (Immunex) and Rubex® (Bristol-Myers Squibb Oncology/Immunology). In one embodiment, the peptide can be a glycopeptide. “Glycopeptide” refers to oligopeptide (e.g. heptapeptide) antibiotics, characterized by a multi-ring peptide core optionally substituted with saccharide groups, such as vancomycin.

Examples of glycopeptides included in this category of antimicrobials may be found in “Glycopeptides Classification, Occurrence, and Discovery,” by Raymond C. Rao and Louise W. Crandall, (“Bioactive agents and the Pharmaceutical Sciences” Volume 63, edited by Ramakrishnan Nagarajan, published by Marcal Dekker, Inc.). Additional examples of glycopeptides are disclosed in U.S. Pat. Nos. 4,639,433; 4,643,987; 4,497,802; 4,698,327, 5,591,714; 5,840,684; and 5,843,889; in EP 0 802 199; EP 0 801 075; EP 0 667 353; WO 97/28812; WO 97/38702; WO 98/52589; WO 98/52592; and in J. Amer. Chem. Soc.(1996) 118: 13107-13108; J. Amer. Chem. Soc. (1997) 119:12041-12047; and J. Amer. Chem. Soc. (1994) 116:4573-4590. Representative glycopeptides include those identified as A477, A35512, A40926, A41030, A42867, A47934, A80407, A82846, A83850, A84575, AB-65, Actaplanin, Actinoidin, Ardacin, Avoparcin, Azureomycin, Balhimyein, Chloroorientiein, Chloropolysporin, Decaplanin, -demethylvancomycin, Eremomycin, Galacardin, Helvecardin, Izupeptin, Kibdelin, LL-AM374, Mannopeptin, MM45289, MM47756, MM47761, MM49721, MM47766, MM55260, MM55266, MM55270, MM56597, MM56598, OA-7653, Orenticin, Parvodicin, Ristocetin, Ristomycin, Synmonicin, Teicoplanin, UK-68597, UD-69542, UK-72051, Vancomycin, and the like. The term “glycopeptide” or “glycopeptide antibiotic” as used herein is also intended to include the general class of glycopeptides disclosed above on which the sugar moiety is absent, i.e. the aglycone series of glycopeptides. For example, removal of the disaccharide moiety appended to the phenol on vancomycin by mild hydrolysis gives vancomycin aglycone. Also included within the scope of the term “glycopeptide antibiotics” are synthetic derivatives of the general class of glycopeptides disclosed above, including alkylated and acylated derivatives. Additionally, within the scope of this term are glycopeptides that have been further appended with additional saccharide residues, especially aminoglycosides, in a manner similar to vancosamine.

The term “lipidated glycopeptide” refers specifically to those glycopeptide antibiotics that have been synthetically modified to contain a lipid substituent. As used herein, the term “lipid substituent” refers to any substituent contains 5 or more carbon atoms, preferably, 10 to 40 carbon atoms. The lipid substituent may optionally contain from 1 to 6 heteroatoms selected from halo, oxygen, nitrogen, sulfur, and phosphorous. Lipidated glycopeptide antibiotics are well known in the art. See, for example, in U.S. Pat. Nos. 5,840,684, 5,843,889, 5,916,873, 5,919,756, 5,952,310, 5,977,062, 5,977,063, EP 667, 353, WO 98/52589, WO 99/56760, WO 00/04044, WO 00/39156, the disclosures of which are incorporated herein by reference in their entirety.

Anti-inflammatory bioactive agents are also useful for dispersion in invention therapeutic polymer compositions. Depending on the body site and disease to be treated, such anti-inflammatory bioactive agents include, e.g. analgesics (e.g., NSAIDS and salicyclates), steroids, antirheumatic agents, gastrointestinal agents, gout preparations, hormones (glucocorticoids), nasal preparations, ophthalmic preparations, otic preparations (e.g., antibiotic and steroid combinations), respiratory agents, and skin & mucous membrane agents. See, Physician 's Desk Reference, 2005 Edition. Specifically, the anti-inflammatory agent can include dexamethasone, which is chemically designated as (11 [symbol] , 16I)-9-fluro-11,17,21-trihydroxy-16-methylpregna-1,4-diene-3,20-dione. Alternatively, the anti-inflammatory bioactive agent can be or include sirolimus (rapamycin), which is a triene macrolide antibiotic isolated from Streptomyces hygroscopicus.

The polypeptide bioactive agents included in the invention compositions and methods can also include “peptide mimetics.” Such peptide analogs, referred to herein as “peptide mimetics” or “peptidomimetics,” are commonly used in the pharmaceutical industry with properties analogous to those of the template peptide (Fauchere, J. (1986) Adv. Bioactive agent Res., 15:29; Veber and Freidinger (1985) TINS, p. 392; and Evans et al. (1987) J. Med. Chem., 30:1229) and are usually developed with the aid of computerized molecular modeling. Generally, peptidomimetics are structurally similar to a paradigm polypeptide (i.e., a polypeptide that has a biochemical property or pharmacological activity), but have one or more peptide linkages optionally replaced by a linkage selected from the group consisting of. —CH₂NH—, —CH₂S—, CH₂—CH₂—, —CH═CH— (cis and trans), —COCH₂—, —CH(OH)CH₂—, and —CH₂SO—, by methods known in the art and further described in the following references: Spatola, A. F. in Chemistry and Biochemistry of Amino Acids, Peptides, and Proteins, B. Weinstein, eds., Marcel Dekker, New York, p. 267 (1983); Spatola, A. F., Vega-Data (March 1983), Vol. 1, Issue 3, “Peptide Backbone Modifications” (general review); Morley, J. S., Trends. Pharm. Sci., (1980) pp. 463-468 (general review); Hudson, D. et al., Int. J. Pept. Prot. Res., (1979) 14:177-185 (—CH₂NH—, CH₂CH₂—); Spatola, A. F. et al., Life Sci., (1986) 38:1243-1249 (—CH₂—S—); Harm, M. M., J Chem. Soc. Perkin Trans I (1982) 307-314 (—CH═CH—, cis and trans); Almquist, R. G. et al., J. Med. Chem., (1980) 23:2533 (—COCH₂—); Jennings-Whie, C. et al., Tetrahedron Lett., (1982) 23:2533 (—COCH₂—); Szelke, M. et al., European Appln., EP 45665 (1982) CA: 97:39405 (1982) (—CH(OH)CH₂—); Holladay, M. W. et al., Tetrahedron Lett., (1983) 24:4401-4404 (—C(OH)CH₂—); and Hruby, V. J., Life Sci., (1982) 31:189-199 (—CH₂—S—). Such peptide mimetics may have significant advantages over natural polypeptide embodiments, including, for example: more economical production, greater chemical stability, enhanced pharmacological properties (half-life, absorption, potency, efficacy, etc.), altered specificity (e.g., a broad-spectrum of biological activities), reduced antigenicity, and others.

Additionally, substitution of one or more amino acids within a peptide (e.g., with a D-Lysine in place of L-Lysine) may be used to generate more stable peptides and peptides resistant to endogenous peptidases. Alternatively, the synthetic polypeptides covalently bound to the biodegradable polymer, can also be prepared from D-amino acids, referred to as inverso peptides. When a peptide is assembled in the opposite direction of the native peptide sequence, it is referred to as a retro peptide. In general, polypeptides prepared from D-amino acids are very stable to enzymatic hydrolysis. Many cases have been reported of preserved biological activities for retro-inverso or partial retro-inverso polypeptides (U.S. Pat. No. 6,261,569 B1 and references therein; B. Fromme et al, Endocrinology (2003)144:3262-3269.

It is readily apparent that the subject invention can be used to prevent or treat a wide variety of diseases or symptoms thereof.

Following preparation of the invention therapeutic polymer compositions and polymer particles thereof, optionally loaded with at least one bioactive agent, the composition can be lyophilized and the dried composition suspended in an appropriate media prior to administration.

Any suitable and effective amount of the at least one bioactive agent can be released with time from the therapeutic polymer composition, including those in a polymer coating on a medical device, such as a stent or a depot formed from particles thereof introduced in vivo. The suitable and effective amount of the bioactive agent will typically depend, e.g., on the specific PEA or PEUR polymer and concentration of therapeutic backbone diol or di-acid incorporated therein, type of particle or polymer/bioactive agent linkage, if present. Typically, up to about 100% of the backbone diol(s) or di-acid(s) and optional bioactive agent(s) can be released from polymer particles sized to avoid circulation as described herein that form a polymer depot in vivo. Specifically, up to about 90%, up to 75%, up to 50%, or up to 25% thereof can be released from the polymer depot. Factors that typically affect the release rate from the polymer depot are the nature and amount of the polymer/backbone therapeutic agent, the types of polymer/bioactive agent linkage, and the nature and amount of additional substances present in the formulation.

Once the invention therapeutic polymer composition is made, as above, the composition is formulated for subsequent intrapulmonary, gastroenteral, subcutaneous, intramuscular, into the central nervous system, intraperitoneum or intraorgan delivery. The compositions will generally include one or more “pharmaceutically acceptable excipients or vehicles” appropriate for oral, mucosal or subcutaneous delivery, such as water, saline, glycerol, polyethylene glycol, hyaluronic acid, ethanol, etc. Additionally, auxiliary substances, such as wetting or emulsifying agents, pH buffering substances, flavorings, and the like, may be present in such vehicles.

For example, intranasal and pulmonary formulations will usually include vehicles that neither cause irritation to the nasal mucosa nor significantly disturb ciliary function. Diluents such as water, aqueous saline or other known substances can be employed with the subject invention. The intrapulmonary formulations may also contain preservatives such as, but not limited to, chlorobutanol and benzalkonium chloride. A surfactant may be present to enhance absorption by the nasal mucosa.

For rectal and urethral suppositories, the vehicle composition will include traditional binders and carriers, such as, cocoa butter (theobroma oil) or other triglycerides, vegetable oils modified by esterification, hydrogenation and/or fractionation, glycerinated gelatin, polyalkaline glycols, mixtures of polyethylene glycols of various molecular weights and fatty acid esters of polyethylene glycol.

For vaginal delivery, the invention therapeutic polymer compositions can be formulated in pessary bases, such as those including mixtures of polyethylene triglycerides, or suspended in oils such as corn oil or sesame oil, optionally containing colloidal silica. See, e.g., Richardson et al., Int J. Pharm. (1995) 115:9-15.

For a further discussion of appropriate vehicles to use for particular modes of delivery, see, e.g., Remington: The Science and Practice of Pharmacy, Mack Publishing Company, Easton, Pa., 19th edition, 1995. One of skill in the art can readily determine the proper vehicle to use for the particular combination of PEA, PEUR or PEU polymer with backbone therapeutic agent or particles thereof and mode of administration.

In addition to humans, the invention therapeutic polymer compositions are also intended as delivery vehicles for use in veterinary administration of bioactive agents to a variety of mammalian patients, such as pets (for example, cats, dogs, rabbits, and ferrets), farm animals (for example, swine, horses, mules, dairy and meat cattle) and race horses.

In one embodiment, the therapeutic polymer compositions used in the invention methods of administration or delivery will comprise an “effective amount” of one or more backbone therapeutic diol or di-acid(s) and optional bioactive agents of interest. That is, an amount of a backbone diol or di-acid will be incorporated into the composition that will produce a sufficient therapeutic or palliative response in order to prevent, reduce or eliminate symptoms. The exact amount necessary will vary, depending on the subject to which the composition is being administered; the age and general condition of the subject; the capacity of the subject's immune system, the degree of therapeutic or palliative response desired; the severity of the condition being treated or investigated; the particular therapeutic diol or di-acid selected and mode of administration of the composition, among other factors. An appropriate effective amount can be readily determined by one of skill in the art. Thus, an “effective amount” will fall in a relatively broad range that can be determined through routine trials. For example, for purposes of the present invention, an effective amount will typically range from about 1 μg to about 100 mg, for example from about 5 μg to about 1 mg, or about 10 μg to about 500 μg of the active agent delivered per dose.

Once formulated, the invention therapeutic polymer compositions are administered orally, mucosally, or by subcutaneously or intramuscular injection, and the like, using standard techniques. See, e.g., Remington: The Science and Practice of Pharmacy, Mack Publishing Company, Easton, Pa., 19th edition, 1995, for mucosal delivery techniques, including intranasal, pulmonary, vaginal and rectal techniques, as well as European Publication No. 517,565 and Illum et al., J. Controlled Rel. (1994) 29:133-141, for techniques of intranasal administration.

Dosage treatment may be a single dose of the invention therapeutic polymer composition, or a multiple dose schedule as is known in the art. The dosage regimen, at least in part, will also be determined by the need of the subject and be dependent on the judgment of the practitioner. Furthermore, if prevention of disease is desired, the therapeutic polymer composition (in the form of particles, or not) is generally administered prior to primary disease manifestation, or symptoms of the disease of interest. If treatment is desired, e.g., the reduction of symptoms or recurrences, the therapeutic polymer compositions are generally administered subsequent to primary disease manifestation.

The formulations can be tested in vivo in a number of animal models developed for the study of oral subcutaneous or mucosal delivery. For example, the conscious sheep model is an art-recognized model for testing nasal delivery of substances See, e.g., Longenecker et al., J. Pharm. Sci. (1987) 76:351-355 and Illum et al., J. Controlled Rel. (1994) 29:133-141. The therapeutic polymer composition, generally in powdered, lyophilized form, is blown into the nasal cavity. Blood samples can be assayed for active agent using standard techniques, as known in the art.

The following examples are meant to illustrate, but not to limit the invention.

### EXAMPLE 1

Materials 17-β-estradiol (estra-1,3,5(10)-triene-3,17β-diol), L-lysine, benzyl alcohol, sebacoyl chloride, 1,6-Hexanediol, p-nitrophenol, triethylamine, 4-N,N-(dimethylamino)pyridine (DMAP), N,N′-dicyclohexylcarbodiimide (DCC), anhydrous N,N-dimethylformamide (DMF), anhydrous dichloromethane (DCM), trifluoroacetic acid (TFA), p-toluenesulfonic acid monohydrate (Aldrich Chemical Co., Milwaukee, Wis.), anhydrous toluene, Boc-L-leucine monohydrate (Calbiochem-Novabiochem, San Diego, Calif.) were used without further purification. Other solvents, ether and ethyl acetate (Fisher Chemical, Pittsburgh, Pa.).

Synthesis of Monomers and Polymers Synthesis of bioactive PEAs involved three basic steps: (1) synthesis of bis(p-nitrophenyl) diesters of dicarboxylic acid (of sebacic acid, compound 1); (2) synthesis of di-p-toluenesulfonic acid salts (or di-TFA salt) of bis(L-leucine) diesters of diol (compounds 2 and 5) and of L-lysine benzyl ester (compound 2); and (3) solution polycondensation of the monomers obtained in steps (1) and (2).

Synthesis of di-p-nitrophenyl esters of sebacic acid (compound 1) Di-p-nitrophenyl ester of sebacic acid was prepared by reacting of sebacoyl chloride with p-nitrophenol as described previously (Katsarava et al. J. Polym. Sci. Part A: Polym. Chem. (1999) 37. 391-407) (Scheme 4):

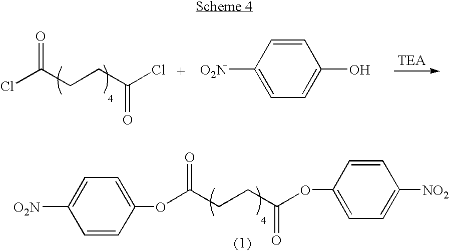

Di-p-toluenesulfonic acid salt of L-lysine benzyl ester (2) was prepared as described earlier (U.S. Pat. No. 6,503,538) by refluxing of benzyl alcohol, toluenesulfonic acid monohydrate and L-lysine monohydro-chloride in toluene, applying azeotropic removal of generated water (scheme 5).

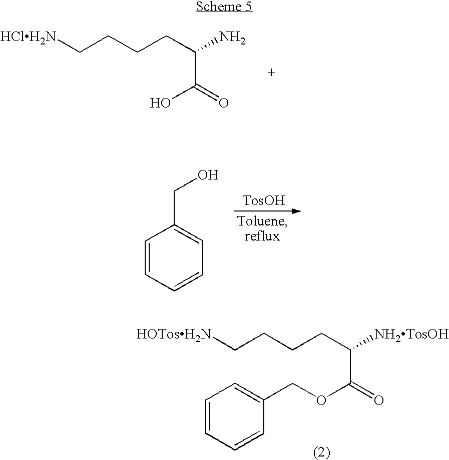

Synthesis of acid salts of bis-(α-amino acid) diesters (3), (5) Di-p-toluenesulfonic acid salt of bis-(L-leucine) hexane-1,6-diester (compound 3) was prepared by modified procedure of the previously published method as shown in scheme 6.

L-Leucine (0.132 mol), p-toluenesulfonic acid monohydrate (0.132 mol) and 1,6-hexanediol (0.06 mol) in 250 mL of toluene were placed in a flask equipped with a Dean-Stark apparatus and overhead stirrer. The heterogeneous reaction mixture was heated to reflux for about 12 h until 4.3 mL (0.24 mol) of water evolved. The reaction mixture was then cooled to room temperature, filtered, washed with acetone, and recrystallized twice from methanol/toluene 2:1 mixture. Yields and Mp were identical to published data (Katsarava et al., supra) (see scheme 6).

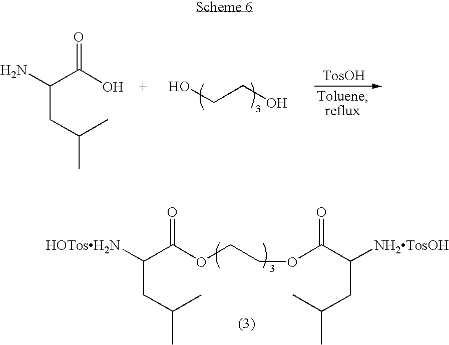

A di-TFA salt of bis-L-leucine-o-estradiol-diester (compound 5) was prepared by a two step reaction. 17-β-Estradiol was first reacted with Boc-protected L-Leucine, applying carbodiimide mediated esterification, to form compound 4. In a second step, Boc groups were deprotected using TFA, converting at the same time into a di-TFA salt of di-amino monomer (compound 5) (see Scheme 7).

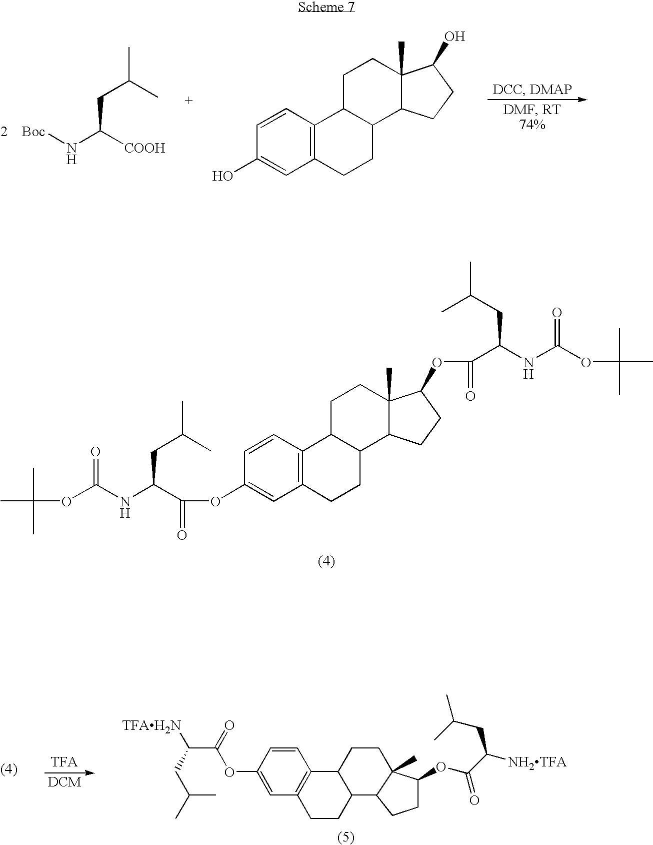

Preparation of Bis(Boc-L-leucine)estradiol-3,17-β-diester (5) 1.5 g (5.51 mmol) of 17-β-estradiol, 3.43 g (13.77 mmol) Boc-L-leucine monohydrate and 0.055 g (0.28 mmol) of p-toluenesulfonic acid monohydrate were dissolved into 20 mL of dry N,N-dimethylformamide at room temperature under a dry nitrogen atmosphere. To this solution 10 g of molecular sieves were added and stirring continued for 24 h. Then, 0.067 g of DMAP and 5.4g of (26.17 mmol) DCC were introduced into the reaction solution and stirring was continued. After 6 h (no discoloration of the reaction was observed), 1 mL of acetic acid was added to destroy the excess of DCC. Precipitated urea and sieves then were filtered off and filtrate poured in 80 mL of water. Product was extracted three times with 30 mL of ethylacetate, dried over sodium sulfate, solvent evaporated, and the product was subjected to chromatography on a column (7:3 hexanes:ethylacetate). A colorless glassy solid of pure compound 4 obtained in a 2.85 g, 74% yield and 100% purity (TLC) and was further converted to compound 5.

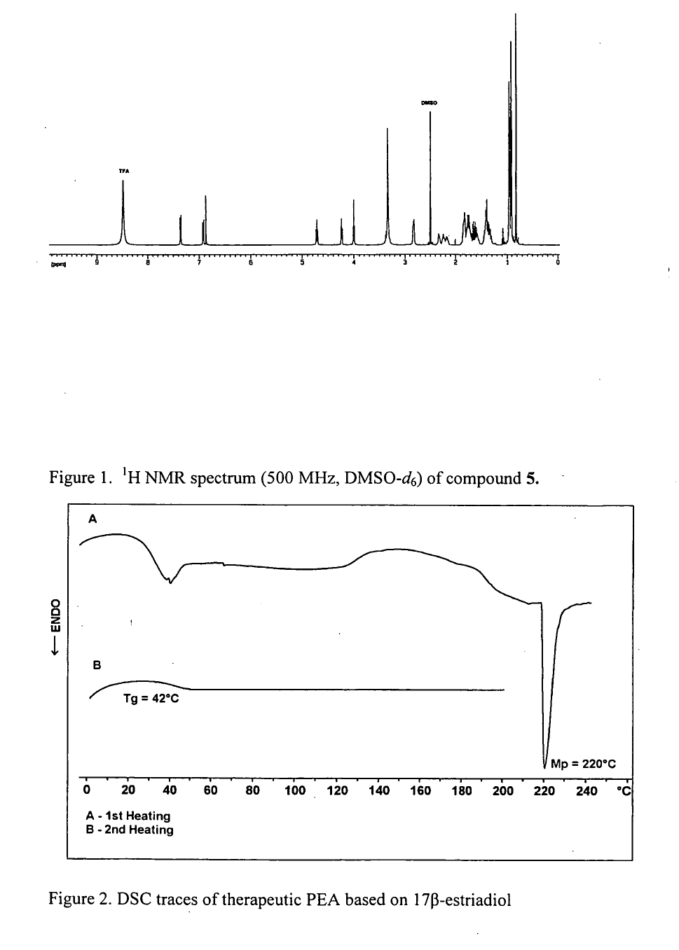

Di-TFA salt of bis(L-leucine)estradiol-3,17-β-diester (compound 5). De-protection of Boc-protected monomer (compound 4) was carried out substantially quantitatively in 10 mL of dry dichloromethane, by adding 4 mL of dry TFA. After 2 h of stirring at room temperature, a homogenous solution was diluted with 300 mL of anhydrous ether and left in a cold room over night. Precipitated white crystals were collected, washed twice with ether, and dried in a vacuum oven at 45° C. Yield 2.67 g (90%). Mp=187.5° C. ¹H NMR (see FIG. 1)

Polymer Synthesis. Synthesis of therapeutic PEA was carried out in DMF in mild conditions (60° C): activated di-acid monomer (compound 1) was reacted with combinations of the di-amino monomers 1.5 eq. (compound 2), 1.5 eq. (compound 5) and 1 eq. of (compound 3).

Triethylamine 1.46 mL (10.47 mmol) was added at once to the mixture of monomers (compound 1) (4.986 mmol), (compound 2) (1.246 mmol), (compound 3) (1.869 mmol), (compound 5) (1.869 mmol) in 3 mL of dry DMF and the solution was heated to 60° C. while stirring. The reaction vial was kept at the same temperature for 16 h. A yellow viscous solution was formed then was cooled down to room temperature, diluted with 9 mL of dry DMF, added 0.2 mL of acetic anhydride, and after 3 h precipitated out three times: first in water, then from ethanol solution into ethylacetate and lastly, from chloroform in ethyl acetate. A colorless hydrophobic polymer was cast as a tough film from chloroform:ethanol (1:1) mixture and dried in vacuum. Yield: 1.74 g (70%)

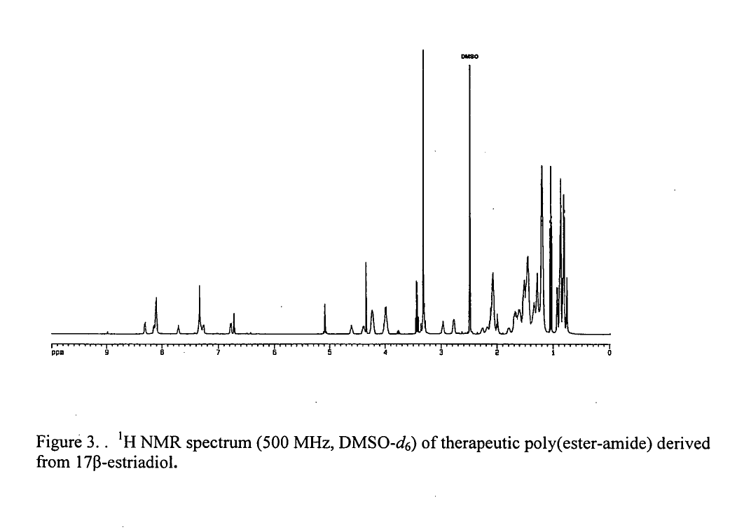

Materials Characterization The chemical structure of monomers and polymer were characterized by standard chemical methods. NMR spectra were recorded by a Bruker AMX-500 spectrometer (Numega R. Labs Inc. San Diego, Calif.) operating at 500 MHz for ¹H NMR spectroscopy. Deuterated solvents CDCl₃ or DMSO-d₆ (Cambridge Isotope Laboratories, Inc., Andover, Mass.) were used with tetramethylsilane (TMS) as internal standard. The results are shown in FIGS. 1 and 3.

Melting points of synthesized monomers were determined on an automatic Mettler-Toledo FP62 Melting Point Apparatus (Columbus, Ohio). Thermal properties of synthesized monomers and polymers were characterized on Mettler-Toledo DSC 822e differential scanning calorimeter. Samples were placed in aluminum pans. Measurements were carried out at a scanning rate of 10° C./min under nitrogen flow (FIG. 2).

The number and weight average molecular weights (Mw and Mn) and molecular weight distribution of synthesized polymer was determined by Model 515 gel permeation chromatography (Waters Associates Inc. Milford, Mass.) equipped with a high pressure liquid chromatographic pump, a Waters 2414 refractory index detector. 0.1% of LiCl solution in N,N-dimethylacetamide (DMAc) was used as eluent (1.0 mL/min). Two Styragel® HR 5E DMF type columns (Waters) were connected and calibrated with polystyrene standards.

Tensile Properties: tensile strength, elongation at break and Young's Modulus were measured on a tensile strength instrument (Chatillon TCD200, integrated with a PC (Nexygen™ FM software)(Chatillon, Largo, Fla.) at a crosshead speed of 100 mm/min. The load capacity was 50 lbs. The film (4×1.6 cm) had a dumbbell shape and thickness of about. 0.125 mm.

Results Four different monomers were copolymerized by polycondensation of activated monomers, affording copoly PEA containing 17% w/w steroid load on a total polymer weight basis. Chemical structure of the product therapeutic polymer composition is shown in scheme 8.

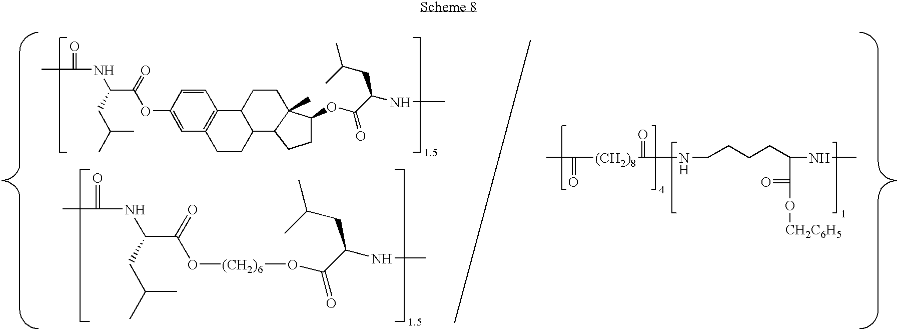

Three monomers: bis-p-toluenesulfonic acid salts of L-lysine-benzyl ester (compound 2), bis(L-leucine) 1,6-hexane diester (compound 3), and bis(p-nitrophenyl) sebacate (compound 1) were prepared according to the literature and characterized by melting point (FIG. 1) and proton NMR spectroscopy (FIG. 3). Results were in agreement with those reported in literature.

In this example a PEA polymer containing a residue of β-Estradiol in the main polymer backbone was prepared, where both hydroxyls of the diol steroid were incorporated into monomer via ester bonds using a carbodiimide technique. The final monomer introduced into the polymerization reaction was a TFA salt. After polycondensation, a high molecular weight copolymer was obtained. Gel permeation chromatography yielded an estimated weight average Mw=82,000 and polydispersity PDI=1.54. The product copolymer was partially soluble in ethanol (when dry), well soluble in chloroform, chloroform:ethanol 1:1 mixture, dichloromethane, and in polar aprotic organic solvents: DMF, DMSO, DMAc.

Glass transition temperature was detected at Tg=41° (midpoint, taken from the second heating curve) and a sharp melting endotherm was detected at 220° C. by Differential scanning calorimetry (DSC) analysis (FIG. 2). This result leads to the conclusion that the polymer has semi-crystalline properties.

The therapeutic polymer formed a tough film when cast from chloroform solution. Tensile characterization yielded the following results: Stress at break 28.1 MPa, Elongation 173%, Young's Modulus 715 MPa.

### EXAMPLE 2

Synthesis of a therapeutic PEUR polymer composition (structural formula IV) containing a therapeutic diol in the polymer backbone is illustrated in this example. A first monomer used in the synthesis is a di-carbonate of a therapeutic diol with a general chemical structure illustrated by formula 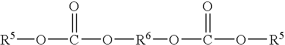
is formed using a known procedure (compound (X) as described in U.S. Pat. No. 6,503,538) wherein R⁵ is independently (C₆-C₁₀) aryl (e.g. 4-nitrophenol, in this example), optionally substituted with one or more nitro, cyano, halo, trifluoromethyl or trifluoromethoxy; and at least some of p-nitrophenol. At least some of R⁶ is a residue of a therapeutic diol as described herein, depending upon the desired drug load. In the case where all of R6 is not the residue of a therapeutic diol, each diol would first be prepared and purified as a separate monomer. For example, di-p-nitrophenyl-3,17-β-estradiol-dicarbonate (compound 6) can be prepared by the method of Scheme 9 below:

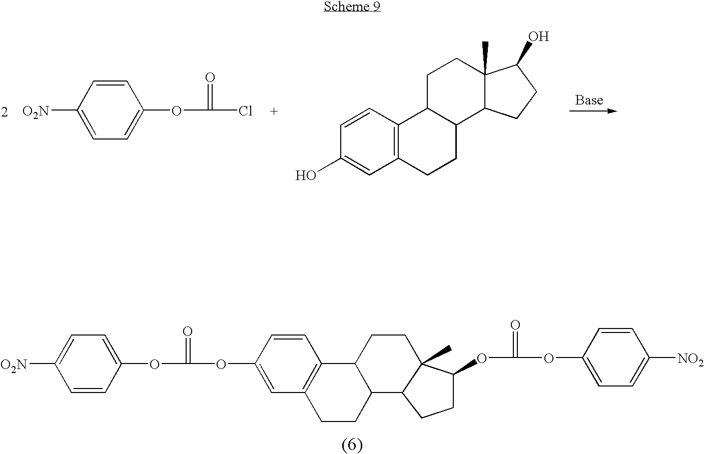

Polycondensation of compound X from U.S. Pat. No. 6,503,538 (in our example compound 6) with the monomers described above yields an estradiol-based co-poly(ester urethane) PEUR (compound 11): 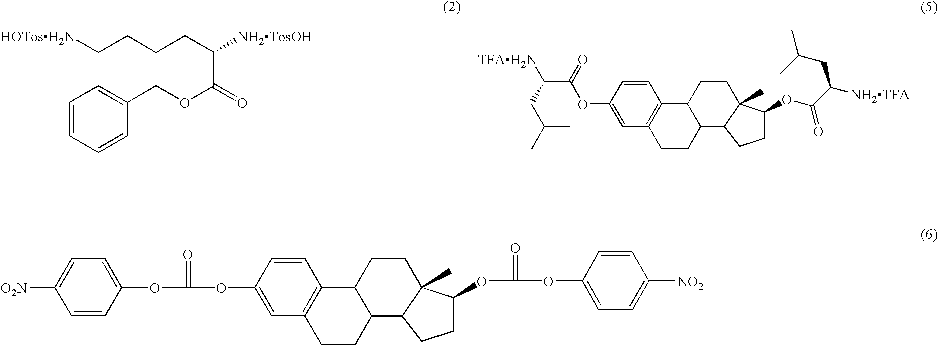
wherein the reaction scheme is as follows

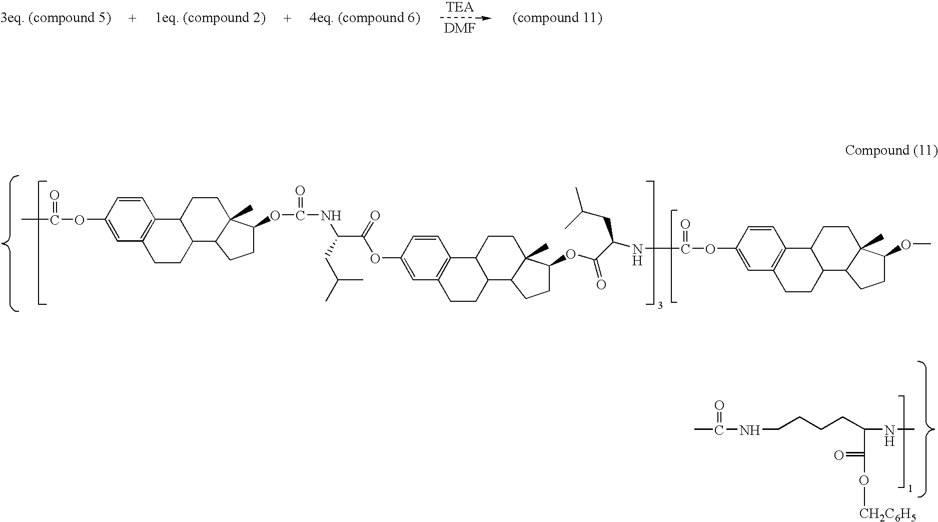

All publications, patents, and patent documents are incorporated by reference herein, as though individually incorporated by reference. The invention has been described with reference to various specific and preferred embodiments and techniques. However, it should be understood that many variations and modifications might be made while remaining within the spirit and scope of the invention.

Although the invention has been described with reference to the above examples, it will be understood that modifications and variations are encompassed within the spirit and scope of the invention. Accordingly, the invention is limited only by the following claims.

What is claimed is:

1. A therapeutic polymer composition comprising at least one therapeutic diol or di-acid bioactive agent incorporated into the backbone of a biodegradable polymer, wherein the polymer comprises or is a blend of at least one poly(ester amide) (PEA) having a chemical formula described by structural formula (I), 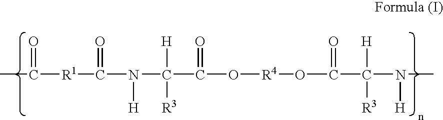
wherein n ranges from about 5 to about 150; R¹ is independently selected from residues of α,ω-bis(4-carboxyphenoxy)-(C₁-C₈) alkane, 3,3′-(alkanedioyldioxy)dicinnamic acid or 4,4′-(alkanedioyldioxy)dicinnamic acid, (C₂-C₂₀) alkylene, (C₂-C₂₀) alkenylene or saturated or unsaturated residues of therapeutic di-acids; the R³s in individual n monomers are independently selected from the group consisting of hydrogen, (C₁-C₆) alkyl, (C₂-C₆) alkenyl, (C₂-C₆) alkynyl, (C₆-C₁₀) aryl (C₁-C₆) alkyl, and —(CH₂)₂S(CH₃); and R⁴ is independently selected from the group consisting of (C₂-C₂₀) alkylene, (C₂-C₂₀) alkenylene, (C₂-C₈) alkyloxy (C₂-C₂₀) alkylene, bicyclic-fragments of 1,4:3,6-dianhydrohexitols of structural formula (II), saturated or unsaturated therapeutic diol residues, and combinations thereof; 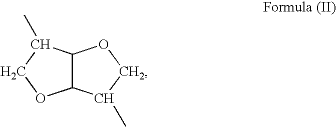
except that at least one of R¹ and R⁴ is a therapeutic amount of the residue of a therapeutic di-acid or diol, respectively,
or at least one PEA polymer having a chemical formula described by structural formula (III): 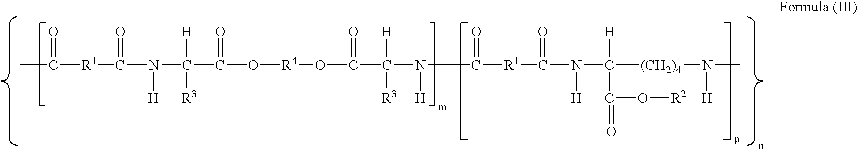
wherein n ranges from about 5 to about 150, m ranges about 0.1 to 0.9: p ranges from about 0.9 to 0.1; wherein R¹ is independently selected from residues of α,ω-bis(4-carboxyphenoxy)-(C₁-C₈) alkane, 3,3′(alkanedioyldioxy)dicinnamic acid or 4,4′(alkanedioyldioxy)dicinnamic acid, (C₂-C₂₀) alkylene, (C₂-C₂₀) alkenylene or a saturated or unsaturated residues of therapeutic di-acids; each R² is independently hydrogen, (C₁-C₁₂) alkyl or (C₆-C₁₀) aryl or a protecting group; the R³s in individual m units are independently selected from the group consisting of hydrogen, (C₁-C₆) alkyl, (C₂-C₆) alkenyl, (C₂-C₆) alkynyl, (C₆-C₁₀) aryl (C₁-C₆) alkyl, and —(CH₂)₂S(CH₃); and R⁴ is independently selected from the group consisting of (C₂-C₂₀) alkylene, (C₂-C₂₀) alkenylene, (C₂-C₈) alkyloxy(C₂-C₂₀) alkylene, bicyclic-fragments of 1,4:3,6-dianhydrohexitols of structural formula (II), residues of saturated or unsaturated therapeutic diols and combinations thereof, except that at least one of R¹ and R⁴ in at least one of the m units is the residue of a therapeutic di-acid or diol, respectively;
or a at least one poly(ester urethane) (PEUR) having a chemical formula described by general structural formula (IV), 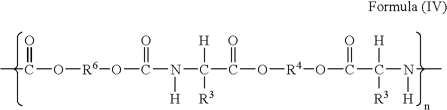
wherein n ranges from about 5 to about 150; wherein R³s in independently selected from the group consisting of hydrogen, (C₁-C₆) alkyl, (C₂-C₆) alkenyl, (C₂-C₆) alkynyl, (C₆-C₁₀) aryl(C₁-C₆) alkyl, and —(CH₂)₂S(CH₃) and; R⁴ is selected from the group consisting of (C₂-C₂₀) alkylene (C₂-C₂₀) alkenylene or alkyloxy, bicyclic-fragments of 1,4:3,6-dianhydrohexitols of structural formula (II), or fragments of saturated or unsaturated therapeutic diols and combinations thereof; and R⁶ is independently selected from (C₂-C₂₀) alkylene, (C₂-C₂₀) alkenylene or alkyloxy, bicyclic-fragments of 1,4:3,6-dianhydrohexitols of general formula (II), residues of saturated or unsaturated therapeutic diols, and combinations thereof, except that the R⁴ and R⁶ within at least one of the n units is the residue of the therapeutic diol;
or at least one PEUR polymer having a chemical structure described by general structural formula (V), 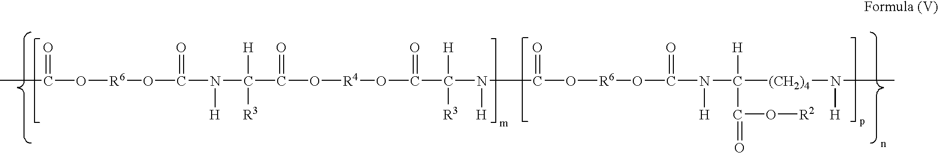
wherein n ranges from about 5 to about 150, m ranges about 0.1 to about 0.9: p ranges from about 0.9 to about 0.1; R² is independently selected from hydrogen, (C₆-C₁₀) aryl (C₁-C₆) alkyl, or a protecting group; the R³s in an individual m unit are independently selected from the group consisting of hydrogen, (C₁-C₆) alkyl, (C₂-C₆) alkenyl, (C₂-C₆) alkynyl, (C₆-C₁₀) aryl(C₁-C₆) alkyl, and —(CH₂)₂S(CH₃); R⁴ is selected from the group consisting of (C₂-C₂₀) alkylene, (C₂-C₂₀) alkenylene or alkyloxy, bicyclic-fragments of 1,4:3,6-dianhydrohexitols of structural formula (II), or fragments of saturated or unsaturated therapeutic diols and combinations thereof; and R⁶ is independently selected from (C₂-C₂₀) alkylene, (C₂-C₂₀) alkenylene or alkyloxy, bicyclic-fragments of 1,4:3,6-dianhydrohexitols of general formula (II), a residue of a saturated or unsaturated therapeutic diol, and combinations thereof, except that the R⁴ and R⁶ within at least one of the m units is the residue of a therapeutic diol,
or at least one poly(ester urea) (PEU) polymer having a chemical formula described by general structural formula (VI), 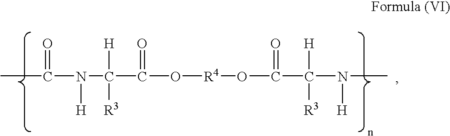
wherein n is about 10 to about 150; each R³s within an individual n monomer are independently selected from hydrogen, (C₁-C₆) alkyl, (C₂-C₆) alkenyl, (C₂-C₆) alkynyl, (C₆-C₁₀) aryl (C₁-C₆)alkyl, and —(CH₂)₂S(CH₃); R⁴ is independently selected from (C₂-C₂₀) alkylene, (C₂-C₂₀) alkenylene, (C₂-C₈) alkyloxy (C₂-C₂₀) alkylene, a residue of a saturated or unsaturated therapeutic diol; or a bicyclic-fragment of a 1,4:3,6-dianhydrohexitol of structural formula (II), and combinations thereof, except that the R⁴ within at least one of the n units is the residue of a therapeutic diol;
or at least one PEU having a chemical formula described by structural formula (VII), 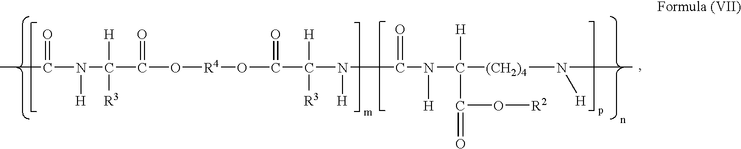
wherein m is about 0.1 to about 1.0; p is about 0.9 to about 0. 1; n is about 10 to about 150; each R² is independently hydrogen, (C₁-C₁₂) alkyl or (C₆-C₁₀) aryl; the R³s within an individual m monomer are independently selected from hydrogen, (C₁-C₆) alkyl, (C₂-C₆) alkenyl, (C₂-C₆) alkynyl, (C₆-C₁₀) aryl (C₁-C₆)alkyl, and —(CH₂)₂S(CH₃); each R⁴ is independently selected from (C₂-C₂₀) alkylene, (C₂-C₂₀) alkenylene, (C₂-C₈) alkyloxy (C₂-C₂₀) alkylene, a residue of a saturated or unsaturated therapeutic diol; or a bicyclic-fragment of a 1,4:3,6-dianhydrohexitol of structural formula (II), and combinations thereof, except that the R⁴ in at least one of the m units is the residue of a therapeutic diol.

2. The composition of claim 1, wherein the polymer has the chemical formula of structural formula (I) and R₃ is CH₂Ph.

3. The composition of claim 1, wherein

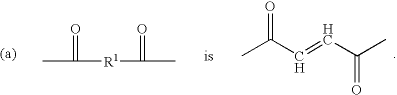

4. The composition of claim 3, wherein RI is selected from —CH₂—CH═CH—CH₂—, —(CH₂)₄—, —(CH₂)₆—, and —(CH₂)₈—.

5. The composition of claim 2, wherein at least one R⁴ is —CH₂—CH═CH—CH₂—.

6. The composition of claim 1, wherein the 1,4:3,6-dianhydrohexitol (II) represents D-glucitol, D-mannitol, or L-iditol.

7. The composition of claim 1, wherein at least one of R¹ or R⁴ is the residue of a therapeutic di-acid or diol respectively.

8. The composition of claim 1, wherein at least one of R⁴ or R⁶ is the residue of a therapeutic diol.

9. The composition of claim 1, where in therapeutic diol is naturally occurring.

10. The composition of claim 1, wherein the therapeutic diol is 17-beta-Estradiol.

11. The composition of claim 1, wherein the therapeutic diol does not occur naturally.

12. The composition of claim 1, wherein at least one R¹ is the residue of a therapeutic di-acid.

13. The composition of claim 1, wherein the composition biodegrades over a period of twenty-four hours, about seven days, about thirty days, or about 90 days.

14. The composition of claim 1, wherein the composition further comprises at least one bioactive agent.

15. The composition of claim 1, wherein the composition includes from about 5 to about 150 molecules of bioactive agent per polymer molecule chain.

16. The composition of claim 15, wherein the at least one bioactive agent is conjugated to the polymer.

17. The composition of claim 1, wherein the polymer of structural formula (III) is contained in a polymer-bioactive agent conjugate having a chemical structure of structural formula (VIII): 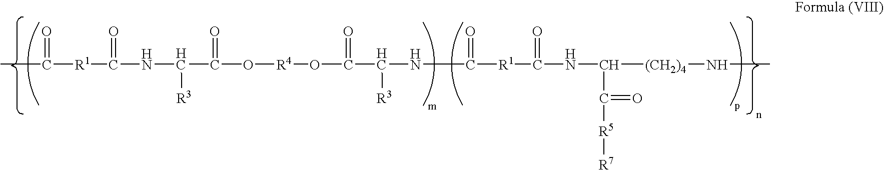
wherein, R⁵ is selected from the group consisting of —O—, —S—, and —NR⁸—; R⁸ is H or (C₁-C₈) alkyl; and R⁷ is the bioactive agent.

18. The composition of claim 17, except that two or more molecules of the polymer composition are crosslinked to provide an —R⁵—R⁷—R⁵ conjugate.

19. The composition of claim 1, wherein the polymer is a PEA of structural formula (I) or (III).

20. The composition of claim 1, wherein the polymer is a PEUR of structural formula (IV) or (V).

21. The composition of claim 1, wherein the polymer is a PEA of structural formula (VI) or (VII).

22. The composition of claim 1, wherein the composition forms a time release polymer depot when administered in vivo.

23. The composition of claim 1, wherein the composition is in the form of disperse droplets containing the particles in a mist.

24. The composition of claim 23, wherein the mist is produced by a nebulizer.

25. The composition of claim 24, wherein the composition is contained within a nebulizer actuatable to produce a mist comprising dispersed droplets of the vehicle.

26. The composition of claim 1, wherein the composition is contained within an injection device that is actuatable to administer the composition by injection.

27. The composition of claim 1, wherein the composition is formulated for administration in the form of a liquid dispersion of the composition.

28. The composition of claim 1, wherein the composition is lyophilized.

29. A method for administering a therapeutic diol or di-acid to a subject by administering to the subject a therapeutic polymer composition of claim 1 in the form of a liquid dispersion, which composition biodegrades by enzymatic action to release the therapeutic diol or di-acid over time.

30. The method of claim 29, wherein the therapeutic diol is a naturally occurring diol.

31. The method of claim 29, wherein the therapeutic diol is 17-beta-Estradiol.

32. The method of claim 29, wherein the therapeutic diol is not naturally occurring.

33. The method of claim 29, wherein the composition is administered by injection.

34. The method of claim 33, wherein the injection is administered intramuscularly, subcutaneously, intravenously, into the Central Nervous System (CNS), into the peritoneum or intraorgan.

35. The method of claim 29, wherein the composition is administered via intrapulmonary or gastroenteral delivery.

36. A bis-nucleophilic compound wherein the compound is a di(amino acid)-estradiol-3,17-β-diester, or salt thereof.

37. The compound of claim 36, wherein the salt is a TFA salt.
